(19) United States
(12) Patent Application Publication
(10) Pub. No.: US 2005/0256150 A1
(43) Pub. Date: Nov. 17, 2005

(54) Enantioselective acyl transfer catalysts and their use in kinetic resolution of alcohols and desymmetrization of meso-diols

(76) Inventor: Vladimir Birman, Brentwood, MO (US)

Correspondence Address: Washington University;Office of Technology Management, 660 S. Euclid Ave., Campus Box 8013, St. Louis, MO, 63110, US

(21) Appl. No.: 11/062,683

(22) Filed: Feb. 22, 2005

Related U.S. Application Data
(60) Provisional application No. 60/546,697, filed on Feb. 20, 2004.

(51) Int. Cl. A61K 31/4745; C07D 471/04

(52) U.S. Cl. 514/292; 514/303; 546/94; 546/118

(57) ABSTRACT

Novel enantioselective acylation catalysts comprising chiral derivatives of DHIP and DHIQ, having the following representative general structures are disclosed: 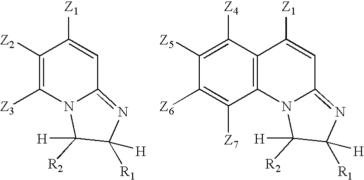 These new compounds are useful for resolving racemates or further enhancing the enantiomeric excess of an enantiomerically enriched composition and for desymmetrizing meso compounds.

### PRIORITY CLAIM TO RELATED PATENT APPLICATION

This patent claims priority to U.S. Provisional Patent Application Ser. No. 60/546,697 filed Feb. 20, 2004. The entire text of that application is incorporated by reference in its entirety into this application.

### FIELD OF THE INVENTION

This invention relates generally to asymmetric catalysis and more particularly relates to novel chiral nucleophilic catalysts for resolving racemates and desymmetrizing meso forms of molecules.

### BACKGROUND OF THE INVENTION

Asymmetric synthesis is of paramount importance to modern organic chemistry, particularly to synthesis of pharmaceuticals. Many bioactive compounds are chiral, and their potencies, pharmacological profiles, and side effects often depend on the absolute configuration of chiral centers in their molecules.

This is why every effort is made to find effective and economical ways of producing chiral compounds used, inter alia, in pharmaceutical industry and perfumery, in the form of pure, individual enantiomers. Catalytic methods are particularly attractive in this respect, since they allow generation or differentiation of chiral centers using only small amounts of chiral catalysts. The following example is illustrative. The widely used antidepressant Fluoxetine (marketed by Eli Lilly as Prozac®) is currently sold in racemic form, although the S-enantiomer is known to be more active (see Robertson, D. W.; Krushinsky, J. H.; Fuller, R. W.; Leander, J. D. Journal of Medicinal Chemistry, 1988, 31, 1412). The S-enantiomer of Fluoxetine can be prepared from the S-enantiomer of 1-phenyl-3-chloropropanol-1 (see, inter alia, Corey, E. J., Reichard, G. A. Tetrahedron Letters, 1989, 30, 5207).

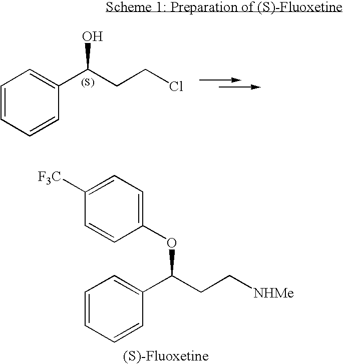

It would be advantageous to isolate the pure S-enantiomer by separating the more easily available racemicl-phenyl-3-chloropropanol-1. However, separation of racemic mixtures into individual enantiomers presents a challenging problem, since all of the physical properties of enantiomers are identical, except for the sign of optical rotation. Thus, standard techniques, such as crystallization, distillation, etc. are not suitable for their separation.

One convenient method of separating racemic mixtures into enantiomers is called kinetic resolution. It is especially economically attractive if it can be accomplished using a catalytic procedure employing only small amounts of an inexpensive chiral catalyst.

In the case of racemic alcohols, kinetic resolution is typically achieved using the so-called asymmetric acyl transfer, or acylation of alcohols with achiral acylating agents in the presence of chiral catalysts.

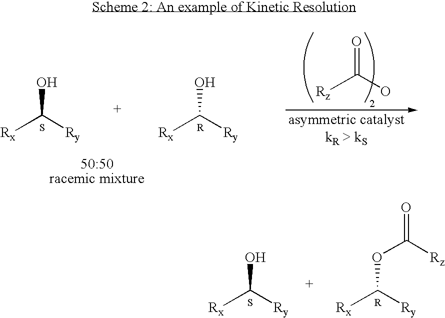

Traditionally, asymmetric acyl transfer has been accomplished by using natural chiral catalysts, or enzymes. However, enzymes, being complex natural compounds, are only available as one of the two possible enantiomers. In addition, there are limitations on the types of substrates that can be resolved and the types of reagents that can be used. For example, enantioselective N-acylation of racemic chiral oxazolidinones, which are important in asymmetric synthesis, has never been accomplished using enzymes.

In recent years, a number of non-enzymatic chiral catalysts were developed which, in some cases, exhibit practically useful levels of enantioselectivity. However, their preparation is typically difficult, often requiring multistep syntheses and laborious resolution of racemates, which is why they have not found widespread use. Structures of representative types of non-enzymatic asymmetric acyl transfer catalysts giving high enantioselectivities are shown in FIG. 1. [See: (a) Ruble, J. C.; Latham, H. A.; Fu, G. J. Am. Chem. Soc. 1997, 119, 1492; (b) Kawabata, T.; Nagato, M.; Takasu, K.; Fuji, K. J. Am. Chem. Soc. 1997, 119, 3169; (c) Spivey, A. C.; Fekner, T.; Spey, S. E. J. Org. Chem. 2000, 65, 3154; (d) Oriyama, T.; Hori, Y.; Imai, K.; Sasaki, R. Tetrahedron Lett. 1996, 37, 8543; (e) Miller, S. J.; Copeland, G. T.; Papaioannou, N.; Horstmann, T.; Ruel, E. M. J. Am. Chem. Soc. 1998, 120, 1629 (f) Vedejs, E.; Daugulis, O.; J. Am. Chem. Soc. 1999, 121, 5813; (g) Ishihara, K.; Kosugi, Y.; Akakura, M. J. Am. Chem. Soc. 2004, 126, 12212.]

[image]

[image]

Thus, despite the existing technology, it is highly desirable to develop novel, inexpensive and versatile catalysts for the kinetic resolution of racemic alcohols and other classes of substrates. In addition, such catalysts can be used in two related asymmetric processes, Dynamic Kinetic Resolution (Scheme 3) and Desymmetrization (Scheme 4).

In Dynamic Kinetic Resolution, the unreacted substrate (e.g., alcohol) undergoes continuous racemization so that eventually all of the racemic mixture is converted into a single enantiomer of the product (illustrated in Scheme 3 as the ester).

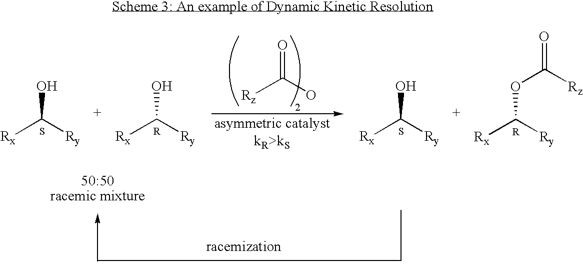

In the Desymmetrization process, the starting material has two chiral moieties of opposite chirality. Such compounds, called meso-compounds (illustrated as a meso-diol in Scheme 4) are not chiral because their molecules have a plane of symmetry. Selective acylation of one of the alcohol moieties removes the symmetry and thus results in a nonracemic chiral product.

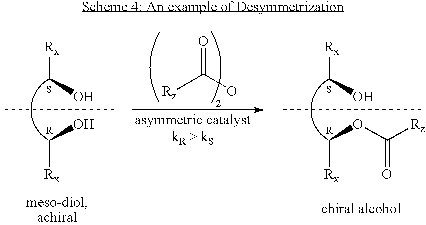

Thus it is highly desirable to develop novel, inexpensive and versatile catalysts for the rapid kinetic resolution of racemic alcohols and desymmetrization of meso-diols at useful yields. Likewise it is highly desirable to have improved processes for the use of such catalysts to kinetically resolve racemic alcohols and desymmetrize meso-diols to form enhanced, enantiomerically enriched products for pharmaceutical and other valuable industrial and commercial uses.

### SUMMARY OF THE INVENTION

This invention relates to novel DHIP (bicyclic basic structure) and DHIQ (tricyclic basic structure) derivative chiral nucleophilic catalyst compounds, and their salts.

The DHIP and DHIQ derivative compounds of the invention have a structure of general formula 1: 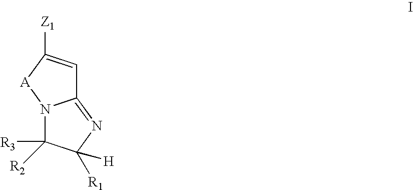
wherein A is selected from the group consisting of: 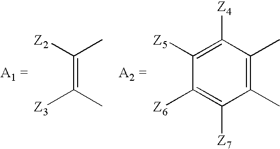
wherein R₁≠H and R₂ and R₃ can be H and in addition R₁, R₂ and R₃ each are independently selected from the group consisting of alkyl, alkenyl, alkynyl, carboaryl, heteroaryl, carbocyclyl, heterocyclyl, oxycarbonyl, aminocarbonyl, each of which can optionally be substituted with one or more substituents independently selected from the group consisiting of (i) unsubstitutable substituents: halogen, cyano, nitro, oxo, and (ii) substitutable substituents: acyl, oxycarbonyl, aminocarbonyl, alkylsulfonyl, arylsulfonyl, aminosulfonyl, hydroxy, acyloxy, alkoxy, aralkoxy, alkylthio, arylthio, aralkylthio, mono- and dialkylamino, mono- and diaralkylamino, cycloamino, carboxamido, sulfamido, trialkylsilyl, alkyl, alkenyl, alkynyl, carboaryl, heteroaryl, carbocyclyl, heterocyclyl; each substitutable substituent of which, in turn, can optionally be substituted with one or more substituents independently selected from the group consisiting of halogen, cyano, nitro, oxo, acyl, oxycarbonyl, aminocarbonyl, alkylsulfonyl, arylsulfonyl, aminosulfonyl, hydroxy, acyloxy, alkoxy, aralkoxy, alkylthio, arylthio, aralkylthio, mono- and dialkylamino, mono- and diaralkylamino, cycloamino, carboxamido, sulfamido, trialkylsilyl, alkyl, alkenyl, alkynyl, carboaryl, heteroaryl, carbocyclyl, heterocyclyl;
wherein R₁ and R₂, and/or R₁ and R₃, and/or R₂ and R₃, can optionally form cyclic structures containing 5 to 10 members wherein any member of the cyclic structure is optionally substituted with one or more substituents independently selected from the group consisiting of (i) unsubstitutable substituents: hydrogen, halogen, cyano, nitro, oxo, and (ii) substitutable substituents: acyl, oxycarbonyl, aminocarbonyl, alkylsulfonyl, arylsulfonyl, aminosulfonyl, hydroxy, acyloxy, alkoxy, aralkoxy, alkylthio, arylthio, aralkylthio, mono- and dialkylamino, mono- and diaralkylamino, cycloamino, carboxamido, sulfamido, trialkylsilyl, alkyl, alkenyl, alkynyl, carboaryl, heteroaryl, carbocyclyl, heterocyclyl; each substitutable substituent of which, in turn, can optionally be substituted with one or more substituents independently selected from the group consisiting of halogen, cyano, nitro, oxo, acyl, oxycarbonyl, aminocarbonyl, alkylsulfonyl, arylsulfonyl, aminosulfonyl, hydroxy, acyloxy, alkoxy, aralkoxy, alkylthio, arylthio, aralkylthio, mono- and dialkylamino, mono- and diaralkylamino, cycloamino, carboxamido, sulfamido, trialkylsilyl, alkyl, alkenyl, alkynyl, carboaryl, heteroaryl, carbocyclyl, heterocyclyl;
wherein Z₁, Z₂, Z₃ Z₄, Z₅, Z₆ and Z₇ are independently selected from the group consisting of (i) unsubstitutable substituents: hydrogen, halogen, cyano, and (ii) substitutable substituents: nitro, arylazo, oxo, acyl, oxycarbonyl, aminocarbonyl, alkylsulfonyl, arylsulfonyl, aminosulfonyl, hydroxy, acyloxy, alkoxy, aralkoxy, alkylthio, arylthio, aralkylthio, mono- and dialkylamino, mono- and diaralkylamino, cycloamino, carboxamido, sulfamido, trialkylsilyl, alkyl, alkenyl, alkynyl, carboaryl, heteroaryl, carbocyclyl, heterocyclyl; each substitutable substituent of which can, in turn, optionally be substituted with one or more substituents independently selected from the group consisiting of halogen, cyano, nitro, arylazo, oxo, acyl, oxycarbonyl, aminocarbonyl, alkylsulfonyl, arylsulfonyl, aminosulfonyl, hydroxy, acyloxy, alkoxy, aralkoxy, alkylthio, arylthio, aralkylthio, mono- and dialkylamino, mono- and diaralkylamino, cycloamino, carboxamido, sulfamido, trialkylsilyl, alkyl, alkenyl, alkynyl, carboaryl, heteroaryl, carbocyclyl, heterocyclyl;
which, in turn, can optionally be substituted with one or more substituents independently selected from the group consisiting of halogen, cyano, nitro, arylazo, oxo, acyl, oxycarbonyl, aminocarbonyl, alkylsulfonyl, arylsulfonyl, aminosulfonyl, hydroxy, acyloxy, alkoxy, aralkoxy, alkylthio, arylthio, aralkylthio, mono- and dialkylamino, mono- and diaralkylamino, cycloamino, carboxamido, sulfamido, trialkylsilyl, alkyl, alkenyl, alkynyl, carboaryl, heteroaryl, carbocyclyl, heterocyclyl,
wherein Z, and Z₂ and/or Z₂ and Z₃ and/or Z, and Z₄ and/or Z₄ and Z₅ and/or Z₅ and Z₆ and/or Z₆ and Z₇ can optionally form cyclic structures containing 5 to 10 members wherein any member of the cyclic structure can optionally be substituted with one or more substituents independently selected from the group consisiting of halogen, cyano, nitro, arylazo, oxo, acyl, oxycarbonyl, aminocarbonyl, alkylsulfonyl, arylsulfonyl, aminosulfonyl, hydroxy, acyloxy, alkoxy, aralkoxy, alkylthio, arylthio, aralkylthio, mono- and dialkylamino, mono- and diaralkylamino, cycloamino, carboxamido, sulfamido, trialkylsilyl, alkyl, alkenyl, alkynyl, carboaryl, heteroaryl, carbocyclyl, heterocyclyl;
provided that when the compound is present in racemic form, and A=A₂ and Z₄, Z₅, Z₆, Z₇, R₂ and R₃ are all H, R₁ cannot be phenyl, 4-fluorophenyl, 4-chlorophenyl, or 2-naphthyl.

In an embodiment of the invention, DHIP novel compounds, and salts thereof, are provided wherein the DHIP derivative compounds have the structure represented by general formula II: 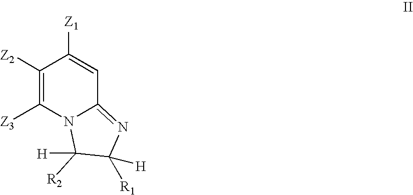
wherein R₁, R₂, Z₁, Z₂ and Z₃ are as defined above.

In a preferred embodiment, the DHIP derivative is a compound of formula II wherein Z₁, Z₃ and R₂ are all H; and
R₁ is selected from the group consisting of branched or unbranched alkyl, cycloalkyl, carboaryl, and heteroaryl, each of which can optionally be substituted with one or more substituents independently selected from the group consisting of halogen, cyano, nitro, acyl, oxycarbonyl, aminocarbonyl, alkylsulfonyl, arylsulfonyl, aminosulfonyl, hydroxyl, acyloxy, alkoxy, aralkoxy, alkylthio, arylthio, aralkylthio, dialkylamino, cycloamino, carboxamido, sulfamido, trialkylsilyl, alkyl, aralkyl, perhaloalkyl, alkenyl, alkynyl, carboaryl, heteroaryl, carbocyclyl, heterocyclyl; and
Z₂ is selected from the group consisting of hydrogen, halogen, cyano, nitro, acyl, oxycarbonyl, aminocarbonyl, alkylsulfonyl, arylsulfonyl, aminosulfonyl, carboxamido, sulfamido, perhaloalkyl, carboaryl, heteroaryl, carbocyclyl, and heterocyclyl.

In a more preferred emobodiment, the DHIP compound has the foregoing definition provided that R₁ is a phenyl group optionally substituted with one or more (up to 5) substituents independently selected from the group consisting of halogen, cyano, nitro, acyl, alkoxycarbonyl, mono- and di-alkylaminocarbonyl, alkylsulfonyl, arylsulfonyl, mono- and di-alkylaminosulfonyl, hydroxyl, acyloxy, alkoxy, aralkoxy, alkylthio, arylthio, aralkylthio, dialkylamino, cycloamino, carboxamido, sulfamido, trialkylsilyl, alkyl, aralkyl, trifluoromethyl, carboaryl, heteroaryl, carbocyclyl, and heterocyclyl.

In a still more preferred embodiment, the DHIP compound has the foregoing definition provided that Z₂ is trifluoromethyl.

In a particularly preferred embodiment, the DHIP has the foregoing definition provided that R₁ is phenyl.

In another embodiment of the invention, DHIQ novel compounds and salts thereof are provided wherein the DHIQ derivative compounds have the structure represented by general formula III: 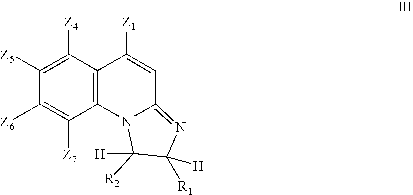
wherein R₁ is not H, and R₃ is H, and in addition R₁, and R₂ each are independently selected from the group consisting of alkyl, alkenyl, alkynyl, carboaryl, heteroaryl, carbocyclyl, heterocyclyl, oxycarbonyl, aminocarbonyl, each of which can optionally be substituted with one or more substituents, each independently selected from the group consisiting of (i) unsubstitutable substituents: halogen, cyano, nitro, oxo, and (ii) substitutable substituents: acyl, oxycarbonyl, aminocarbonyl, alkylsulfonyl, arylsulfonyl, aminosulfonyl, hydroxy, acyloxy, alkoxy, aralkoxy, alkylthio, arylthio, aralkylthio, mono- and dialkylamino, mono- and diaralkylamino, cycloamino, carboxamido, sulfamido, trialkylsilyl, alkyl, alkenyl, alkynyl, carboaryl, heteroaryl, carbocyclyl, heterocyclyl; wherein each of the substitutable subsituents, in turn, can optionally be substituted with one or more substituents independently selected from the group consisiting of halogen, cyano, nitro, oxo, acyl, oxycarbonyl, aminocarbonyl, alkylsulfonyl, arylsulfonyl, aminosulfonyl, hydroxy, acyloxy, alkoxy, aralkoxy, alkylthio, arylthio, aralkylthio, mono- and dialkylamino, mono- and diaralkylamino, cycloamino, carboxamido, sulfamido, trialkylsilyl, alkyl, alkenyl, alkynyl, carboaryl, heteroaryl, carbocyclyl, heterocyclyl;
wherein Z₁, Z₄, Zs, Z₆ and Z₇ are each independently selected from the group consisting of (i) unsubstitutable substituents: halogen, cyano, nitro, arylazo, oxo, and (ii) substitutable substituents: hydrogen, acyl, oxycarbonyl, aminocarbonyl, alkylsulfonyl, arylsulfonyl, aminosulfonyl, hydroxy, acyloxy, alkoxy, aralkoxy, alkylthio, arylthio, aralkylthio, mono- and dialkylamino, mono- and diaralkylamino, cycloamino, carboxamido, sulfamido, trialkylsilyl, alkyl, alkenyl, alkynyl, carboaryl, heteroaryl, carbocyclyl, heterocyclyl; each substitutable substituent of which can optionally be substituted with one or more substituents independently selected from the group consisiting of halogen, cyano, nitro, arylazo, oxo, acyl, oxycarbonyl, aminocarbonyl, alkylsulfonyl, arylsulfonyl, aminosulfonyl, hydroxy, acyloxy, alkoxy, aralkoxy, alkylthio, arylthio, aralkylthio, mono- and dialkylamino, mono- and diaralkylamino, cycloamino, carboxamido, sulfamido, trialkylsilyl, alkyl, alkenyl, alkynyl, carboaryl, heteroaryl, carbocyclyl, heterocyclyl; wherein each substitutable substituent of which, in turn, can optionally be substituted with one or more substituents independently selected from the group consisiting of halogen, cyano, nitro, arylazo, oxo, acyl, oxycarbonyl, aminocarbonyl, alkylsulfonyl, arylsulfonyl, aminosulfonyl, hydroxy, acyloxy, alkoxy, aralkoxy, alkylthio, arylthio, aralkylthio, mono- and dialkylamino, mono- and diaralkylamino, cycloamino, carboxamido, sulfamido, trialkylsilyl, alkyl, alkenyl, alkynyl, carboaryl, heteroaryl, carbocyclyl, and heterocyclyl.

In a preferred embodiment, the DHIQ compound has the immediately foregoing definition provided that Z₄, Z₇, and R₂ are all H; and
R₁ is selected from the group consisting of branched or unbranched alkyl, cycloalkyl, carboaryl and heteroaryl, each of which can optionally be substituted with one or more substituents independently selected from the group consisiting of halogen, cyano, nitro, acyl, oxycarbonyl, aminocarbonyl, alkylsulfonyl, arylsulfonyl, aminosulfonyl, hydroxy, acyloxy, alkoxy, aralkoxy, alkylthio, arylthio, aralkylthio, dialkylamino, cycloamino, carboxamido, sulfamido, trialkylsilyl, alkyl, aralkyl, perhaloalkyl, including, but not limited to, trifluoromethyl, alkenyl, alkynyl, carboaryl, heteroaryl, carbocyclyl, heterocyclyl; and
Z₁, Z₅, and Z₆ are independently selected from the group consisting of (i) unsubstitutable substituents: halogen, cyano, nitro, and (ii) substitutable substituents: hydrogen, acyl, oxycarbonyl, aminocarbonyl, alkylsulfonyl, arylsulfonyl, aminosulfonyl, carboxamido, sulfonamido, perhaloalkyl, including, but not limited to, trifluoromethyl, carboaryl, heteroaryl, carbocyclyl, heterocyclyl;
wherein the substitutable substituents can optionally be substituted with one or more substituents independently selected from the group consisiting of halogen, cyano, nitro, acyl, oxycarbonyl, aminocarbonyl, alkylsulfonyl, arylsulfonyl, aminosulfonyl, hydroxy, acyloxy, alkoxy, aralkoxy, alkylthio, arylthio, aralkylthio, dialkylamino, cycloamino, carboxamido, sulfonamido, trialkylsilyl, alkyl, aralkyl, perhaloalkyl, including, but not limited to, trifluoromethyl, alkenyl, alkynyl, carboaryl, heteroaryl, carbocyclyl, heterocyclyl; and
wherein Z₅ and Z₆ can optionally jointly form a carbocyclic, heterocyclic, aromatic or heteroaromatic cyclic structure containing 5 to 7 members wherein any member of the cyclic structure can optionally be substituted with one or more substituents independently selected from the group consisiting of halogen, cyano, nitro, acyl, oxycarbonyl, aminocarbonyl, alkylsulfonyl, arylsulfonyl, aminosulfonyl, hydroxy, acyloxy, alkoxy, aralkoxy, alkylthio, arylthio, aralkylthio, dialkylamino, cycloamino, carboxamido, sulfamido, trialkylsilyl, alkyl, aralkyl, perhaloalkyl, including, but not limited to, trifluoromethyl, alkenyl, alkynyl, carboaryl, heteroaryl, carbocyclyl, heterocyclyl.

In a more preferred embodiment, the DHIQ compound has the foregoing definition provided that Z₁, Z₄, Z₆, Z₇ and R₂ are all H.

In a still more preferred embodiment, the DHIQ compound is as defined immediately above, provided that: R₁ is a phenyl group optionally substituted with one or more (up to 5) substituents independently selected from the group consisiting of halogen, cyano, nitro, acyl, alkoxycarbonyl, mono- and dialkylaminocarbonyl, alkylsulfonyl, arylsulfonyl, mono- and dialkylaminosulfonyl, hydroxy, acyloxy, alkoxy, aralkoxy, alkylthio, arylthio, aralkylthio, dialkylamino, cycloamino, carboxamido, sulfamido, trialkylsilyl, alkyl, aralkyl, trifluoromethyl, carboaryl, heteroaryl.

In a more particularly preferred embodiment, the DHIQ compound is as defined immediately above, provided that: Z₂ is halogen.

In a particularly preferred embodiment, the DHIQ compound is as defined immediately above, provided that: R₁ is phenyl and Z₂ is chlorine.

In a principal aspect, the invention relates to novel compounds useful for resolving racemic mixtures or further enhancing an enatiomeric excess of an already enantiomerically enriched chiral substrate, and desymmtrizing meso-alcohols. In another principal aspect, the invention relates to novel compounds that are easily prepared from commercially available starting materials and structural modifications and for enhancing properties of the compounds according to specific needs are accomplished without undue difficulty.

In yet another aspect, the invention relates to methods for catalyzing reactions comprising forming a catalyzable composition comprising at least one DHIP or DHIQ asymmetric nucleophilic catalyst and at least one of a racemic alcohol or a meso-molecule composition and contacting the catalyzable composition for a suitable time and at an effective temperature to form a nonracemic alcohol or desymmetrized meso-diol composition and then recovering the desired product from the catalyzed composition and refining or purifying the product as needed.

### DETAILED DESCRIPTION OF THE INVENTION

Novel asymmetric nucleophilic catalysts providing high levels of enantionselectivity in kinetic resolution of racemic secondary alcohols and meso-diols are disclosed. The novel asymmetric acyl transfer catalysts are both effective and easily synthesized in numerous structural variations. Further, a new method of preparing these novel catalysts and a new method for the kinetic resolution of racemic secondary alcohols (also referred to herein as secondary alcohol substrates) and meso-diols is disclosed.

The novel, effective enantioselective acylation catalysts (hereinafter referred to “DHIP” of “DHIQ” derivatives) establish the utility of the DHIP and DHIQ derivatives for asymmetric catalysis, and the more general discovery of their catalytic activity for other reactions. In particular, the novelty and utility of these novel chiral derivatives of DHIP and DHIQ for resolving racemic secondary alcohols using a number of different substrates, such as secondary alcohol substrates, are confirmed.

A difference in the reactivity of enantiomers towards the novel catalysts occurs in this kinetic resolution reaction process. This difference in reactivity results in preferential transformation of one enantiomer into a new product, while the other enantiomer remains largely unchanged. Kinetic resolutions and desymmetrizations can be accomplished by using at least one of these novel stoichiometric resolving agents.

The new product and the unreacted starting material possess different physical properties and therefore can be separated and recovered if desired. This allows optional separation and recovery of a desired nonracemic secondary alcohol or a desired desymmetrized meso-diol.

### Chemistry

Synthesis

Generally reactions employed to prepare catalysts of this discovery and to use catalysts of this discovery are carried out in a suitable reaction system such as a vessel, reactor or other sufficient container having suitable capable means for providing reaction conditions such as temperature, mixing and time necessary to bring about the desired catalytic reaction and reaction products. Suitable mixing means such as a stirrer are provided in situations where a stirrer may be advantageously employed.

The stoichiometry is generally selected so that the desired reaction proceeds successftilly.

As used herein, by “substituted” as in “substituted hydrocarbyl,” “substituted hydrocarbylene,” “substituted alkyl,” “substituted alkenyl” and the like, as alluded to in some of the aforementioned definitions, is meant that in the hydrocarbyl, hydrocarbylene, alkyl, alkenyl or other moiety, at least one hydrogen atom bound to a carbon atom is replaced with one or more substituents that are functional groups such as hydroxyl, alkoxy, thio, amino, halo, silyl, and the like. When the term “substituted” appears prior to a list of possible substituted groups, it is intended that the term apply to every member of that group. That is, the phrase “substituted alkyl, alkenyl and alkynyl” is to be interpreted as “substituted alkyl, substituted alkenyl and substituted alkynyl.” Similarly, “optionally substituted alkyl, alkenyl and alkynyl” is to be interpreted as “optionally substituted alkyl, optionally substituted alkenyl and optionally substituted alkynyl.”

The following terms have the meanings assigned to them throughout the specification and claims:

Terms and Definitions

The term “chirality” shall mean the geometric property of a rigid object (or spatial arrangement of points or atoms) of being non-superposable on its mirror image; such an object has no symmetry elements of the second kind (a mirror plane, s=S1, a centre of inversion, i=S2, a rotation-reflection axis, S2n). A carbon atom is said to be “chiral” if all four substituents attached to it are different. See IUPAC Compendium of Chemical Terminology 2nd Edition (1997).

The term “achiral” shall mean that the object is superposable on its mirror image. See IUPAC Compendium of Chemical Terminology 2nd Edition (1997)

The term “catalysis” shall mean the ability of certain compounds (catalysts), typically used in small amounts, to facilitate (catalyze) chemical reactions without being consumed in the course of the reaction.

The term “racemic or racemate” shall mean an equimolar mixture of a pair of enantiomers. It does not exhibit optical activity. The chemical name or formula of a racemate is distinguished from those of the enantiomers by the prefix (O)- or rac-(or racem-) or by the symbols RS and SR. See IUPAC Compendium of Chemical Terminology 2^{nd} Edition (1997).

The term “Racemic mixture”, “racemic composition”, “racemic”, “racemate” and “(±)” terminology are used interchangeably herein.

The term “enantiomer” shall mean one of a pair of molecular entities which are mirror images of each other and non-superposable. See IUPAC Compendium of Chemical Terminology 2nd Edition (1997).

The term “enantiomer excess” or “enantiomeric excess” or “e.e.” shall mean for a mixture of (+)- and (−)-enantiomers, with composition given as the mole or weight fractions F(+) and F(−) (where F(+)+F(−)=1) the enantiomer excess is defined as IF(+)-F(−)1 (and the percent enantiomer excess by 100|F(+)−F(−)|) See IUPAC Compendium of Chemical Terminology 2nd Edition (1997).

The term “enantiomerically pure” shall mean only one of the enantiomers can be detected by analytical methods.

The term “enantioenriched composition” (alone or in combination with another term(s)) shall mean a compostion of a chial substance whose enantiomeric ratio is greater than 50:50 but less than 100:0. See IUPAC Compendium of Chemical Terminology, “Goldbook”, Second Edition, 1997.

The term “enantiopure composition” shall mean a composition containing molecules all having the same chirality sense (within the limits of detection). See IUPAC Compendium of Chemical Terminology, “Goldbook”, Second Edition, 1997.

The term “enantioselective” refers to a process or reaction, which creates an excess of one enantiomer of a pair. (F. A. Carey, R. J. Sundberg “Advanced Organic Chemistry” Part A. 3^{rd} Edn. Plenum Press 1990)

The term “optically active” shall mean mixtures containing more of one enantiomer than the other. It can also be called as “nonracemic”. See IUPAC Compendium of Chemical Terminology 2nd Edition (1997).

The term “kinetic resolution” shall mean the achievement of partial or complete resolution by virtue of unequal rates of reaction of the enantiomers in a racemate with a chiral agent (reagent, catalyst, solvent, etc.). See IUPAC Compendium of Chemical Terminology 2nd Edition (1997).

The term “asymmetric catalysis” refers to the ability of certain chiral compounds (asymmetric catalysts) to catalyze reactions leading to predominant production of one enantiomer over the other, or reactions transforming one enantiomer in a mixture in preference to the other. The latter reaction forms the basis for catalytic kinetic resolution (or catalytic desymmetrization).

The term “desymmetrization” is defined as a process by which one of the two chiral centers of opposite Chirality present in a meso-compound is chemically modified in preference to the other, thereby creating a non-symmetrical (chiral) molecule in a non-racemic form.

The term “meso-” refers to a compound which contains chiral centers but is nevertheless achiral due to the presence of a plane of symmetry. Such a situation occurs when the chiral centers have opposite chiralities and therefore can be reflected onto each other.

The term “acylation” shall mean a process by which a substituent, most typically hydrogen, is replaced by an acyl group.

The term “Hydrocarbyl” is defined as a substituent which consists of carbon and hydrogen atoms, wherein the carbon atoms are connected by means of single and/or double and/or triple bonds and can optionally form aromatic rings.

The term “alkyl” (alone or in combination with another term(s)) is intended to mean a straight-or branched-chain saturated hydrocarbyl substituent typically containing from 1 to about 20 carbon atoms, more typically from 1 to about 8 carbon atoms, and even more typically from 1 to about 6 carbon atoms. Examples of such substituents include methyl, ethyl, n-propyl, isopropyl, n-butyl, isobutyl, sec-butyl, tert-butyl, pentyl, iso-amyl, hexyl, octyl, and the like.

The term “alkenyl” (alone or in combination with another term(s)) is intended to mean a straight- or branched-chain hydrocarbyl substituent containing one or more double bonds and typically from 2 to about 20 carbon atoms, more typically from about 2 to about 8 carbon atoms, and even more typically from about 2 to about 6 carbon atoms. Examples of such substituents include ethenyl (vinyl); 1-propenyl; 2-propenyl; 1,4-pentadienyl; 1,4-butadienyl; 1-butenyl; 2-butenyl; 3-butenyl; 5-decenyl; and the like.

The term “alkynyl” (alone or in combination with another term(s)) is intended to mean a straight- or branched-chain hydrocarbyl substituent containing one or more triple bonds and typically from 2 to about 20 carbon atoms, more typically from about 2 to about 8 carbon atoms, and even more typically from about 2 to about 6 carbon atoms. Examples of such substituents include ethynyl, 1-propynyl, 2-propynyl, 4-decynyl, 1-butynyl, 2-butynyl, 3-butynyl, and the like.

The term “aryl” (alone or in combination with another term(s)) is intended to mean a carbocyclyl or heterocyclyl containing at least one aromatic ring and from a 5 to 14 carbon ring atoms. Examples of aryls include both carboaryls, such as phenyl, naphthyl, and indenyl, and heteroaryls, such as pyridyl, quinolinyl indolyl, furyl, thienyl etc.

The term “hydrogen” shall mean hydrogen radical which may be depicted as —H.

The term “hydroxy” (alone or in combination with another term(s)) shall mean —OH.

The term “nitro” (alone or in combination with another term(s)) shall mean—NO₂.

The term “cyano” (alone or in combination with another term(s)) shall means —CN.

The term “carboxy” (alone or in combination with another term(s)) shall means —C(OFH.

The term “amino” (alone or in combination with another term(s)) shall mean —NH₂—. The term “monosubstituted amino” (alone or in combination with another term(s)) means an amino substituent wherein one of the hydrogen radicals is replaced by a non-hydrogen substituent. The term “disubstituted amino” (alone or in combination with another term(s)) means an amino substituent wherein both of the hydrogen atoms are replaced by non-hydrogen substituents, which may be identical or different.

The term “cycloamino” (alone or in combination with another term(s)) is intended to mean a disubstituted amino wherein both of the non-hydrogen substituents, which may be identical or different, together form a ring structure containing 3 to 10 members.

The term “oxo” is intended to mean an oxygen atom connected by a double bond to a carbon atom. For example, 2-oxopropyl means —CH₂—C(O)—CH₃.

The term “acyl” is intended to mean a substituent which may be depicted as —C(O)—Y, where Y can be either hydrogen or a non-hydrogen substituent.

The term “acyloxy” is intended to mean a substituent which may be depicted as —O—C(O)—Y, where Y can be either hydrogen or a non-hydrogen substituent.

The term “aralkyl” is intended to mean an alkyl substituent substituted with at least one aryl group and optionally substituted with other aryl or other non-hydrogen substituents.

The term “halogen” (alone or in combination with another term(s)) shall mean a fluorine radical (which may be depicted as —F), chlorine radical (which may be depicted as —Cl), bromine radical (which may be depicted as —Br), or iodine radical (which may be depicted as −1).

The term “sulfonamido” is intended to mean a substituent which may be depicted as —N(Y)—SO₂—X, wherein X is a non-hydrogen substituent and Y can be either hydrogen or a non-hydrogen substituent.

The term “carbonyl” (alone or in combination with another term(s)) shall mean —C(O)—. This term also is intended to encompass a hydrated carbonyl substituent, i.e., —C(OH)₂—.

The term “aminocarbonyl” (alone or in combination with another term(s)) is intended to mean —C(O)—NH₂, wherein one or both of the H are optionally substituted by non-hydrogen substituents.

The term “oxy” (alone or in combination with another term(s)) means an ether substituent, and may be depicted as —O—.

The term “alkoxy” (alone or in combination with another term(s)) shall mean an alkylether substituent, i.e., —O-alkyl. Examples of such a substituent include methoxy (—O—CH₃), ethoxy, n-propoxy, isopropoxy, n-butoxy, iso-butoxy, sec-butoxy, tert-butoxy, and the like.

The term “thio” (alone or in combination with another term(s)) is intended to mean a thiaether substituent, i.e., an ether substituent wherein a divalent sulfur atom is in the place of the ether oxygen atom. Such a substituent may be depicted as —S—. Thus, for example, “alkyl-thio-alkyl” means alkyl-5-alkyl.

It is understood that wherever the term “thio” or “alkylthio” or “arylthio” or “aralkylthio” and the like are used to describe a moiety that can be represented as “Substituent-S-Substituent”, the derivatives “Substituent-S(OYSubstituent”, called sulfoxides, and “Substituent-S(O)₂-Substituent”, called sulfones, are also implied.

The term “sulfonyl” (alone or in combination with another term(s)) is intended to mean —S(O)₂—. Thus, for example, “aryl-sulfonyl-alkyl” means aryl-S(O)₂-alkyl-.

The term “aminosulfonyl” (alone or in combination with another term(s)) is intended to mean —S(O)₂—NH₂, wherein one or both of the H are optionally substituted by non-hydrogen substituents.

The term “heterocyclyl” means a substituent containing in its structure at least one ring wherein at least one of the ring atoms is a heteroatom, such as oxygen, sulfur, or nitrogen, with the remaining atoms being independently carbons or heteroatoms. A heterocyclyl may have a single ring, which typically contains from 3 to 7 ring atoms, more typically 5 to 6 ring atoms, or several rings, typically 2 or 3, each of which is typically composed of 3 to 7 ring atoms, more typically 5 to 6 ring atoms. The term “heterocyclyl” encompasses saturated, partially unsaturated, or aromatic (i.e., heteroaryl) structures. When there are two or more rings present, they can be fused to one another or form a bridged system.

Examples of single-ring heterocyclyls include oxiranyl, aziridinyl, oxetanyl, azetidinyl, tetrahydrofuryl, tetrahydrothienyl, pyrrolidinyl, oxazolidinyl, thiazolidinyl, imidazolidinyl, tetrahydropyranyl, piperidinyl, morpholinyl, piperazinyl, hexahydroazepinyl, dihydrofuryl, pyrrolinyl, oxazolinyl, thiazolinyl, imidazolinyl, tetrahydropyridyl, furyl, thienyl, pyrrolyl, oxazolyl, thiazolyl, isoxazolyl, isothiazolyl, pyrazolyl, imidazolyl, triazolyl, tetrazolyl, oxadiazolyl, thiadiazolyl, pyridyl, pyrimidinyl, pyrazinyl, pyridazinyl, triazinyl,

Examples of bicyclic and tricyclic heterocyclyls include azanorbornyl, tropanyl, perhydroindolyl, indolyzidinyl, quinolyzidinyl, indolinyl, isoindolinyl, dihydrobenzopyranyl, tetrahydroquinolinyl, benzofuranyl, thionaphthenyl, indolyl, benzoxazolyl, benzothiazolyl, benzimidazolyl, indazolyl, benzotriazolyl, indolyzinyl, quinolinyl, isoquinolinyl, quinazolinyl, quinoxalinyl, phthalazinyl, cinnolinyl, quinolyzinyl, imidazopyridyl, purinyl, dibenzofuranyl, dibenzothiophenyl, carbazolyl, carbolinyl, xanthenyl, thioxanthenyl, acridinyl, dibenzodioxinyl, phenoxazinyl, phenothiazinyl, phenazinyl, phenanthridinyl,

The term “carbocyclyl” means a substituent containing in its structure at least one ring wherein all of the ring atoms are carbons. A carbocyclyl may have a single ring, which typically contains from 3 to 7 ring atoms, more typically 5 to 6 ring atoms, or several rings, typically 2 or 3, each of which is typically composed of 3 to 7 ring atoms, more typically 5 to 6 ring atoms. The term “carbocyclyl” encompasses saturated, partially unsaturated, or aromatic (i.e., carboaryl) structures. When there are two or more rings present, they can be fused to one another or form a bridged system.

Examples of single-ring carbocyclyls include cyclopropyl, cyclobutyl, cyclopentyl, cyclohexyl, cycloheptyl, cyclopentenyl, cyclohexenyl, cycloheptenyl, cyclopentadienyl, phenyl. Examples of bicyclic and tricyclic carbocyclyls include, without limitation, norbornyl, hydrindanyl, decalinyl, norbornenyl, norbornadienyl, indanyl, tetralinyl, naphthyl, fluorenyl, anthracenyl, phenanthryl, ferrocenyl.

The term “carboaryl” is intended to mean a substituent containing in its structure at least one aromatic ring wherein all of the ring atoms are carbons. A carboaryl may have a single ring, which typically contains from 5 to 6 ring atoms, more typically 6 ring atoms (i.e., benzene ring), or several rings, typically 2 or 3, each of which is typically composed of 5 to 6 ring atoms, more typically 6 ring atoms. Examples of carboaryls include phenyl, indanyl, tetralinyl, naphthyl, fluorenyl, anthracenyl, phenanthryl, ferrocenyl.

The term “heteroaryl” is intended to mean a substituent containing in its structure at least one aromatic ring wherein at least one of the ring atoms is a heteroatom, such as oxygen, sulfur, or nitrogen with the remaining atoms being independently carbons or heteroatoms. A heteroaryl may have a single ring, which typically contains from 5 to 6 ring atoms, or several rings, typically 2 or 3, each of which is typically composed of 5 to 6 ring atoms. Examples of heteroaryls include furyl, thienyl, pyrrolyl, oxazolyl, thiazolyl, isoxazolyl, isothiazolyl, pyrazolyl, imidazolyl, triazolyl, tetrazolyl, oxadiazolyl, thiadiazolyl, pyridyl, pyrimidinyl, pyrazinyl, pyridazinyl, triazinyl, benzofuranyl, thionaphthenyl, indolyl, benzoxazolyl, benzothiazolyl, benzimidazolyl, indazolyl, benzotriazolyl, indolyzinyl, quinolinyl, isoquinolinyl, quinazolinyl, quinoxalinyl, phthalazinyl, cinnolinyl, quinolyzinyl, imidazopyridyl, purinyl, dibenzofuranyl, dibenzothiophenyl, carbazolyl, carbolinyl, acridinyl, phenazinyl, phenanthridinyl.

The term “cycloalkyl” is intended to mean a fully saturated carbocyclyl substituent. A cycloalkyl may have a single ring, which typically contains from 3 to 7 ring atoms, more typically 5 to 6 ring atoms, or several rings, typically 2 or 3, each of which is typically composed of 3 to 7 ring atoms, more typically 5 to 6 ring atoms. When there are two or more rings present, they can be fused to one another or form a bridged system. Examples of cycloalkyls include cyclopropyl, cyclobutyl, cyclopentyl, cyclohexyl, cycloheptyl, norbornyl, hydrindanyl, decalinyl,

The term “substitutable” shall mean the substituent containing one or more hydrogen atoms. Thus, for example, halogen, nitro, oxo and cyano groups do not fall within this definition.

If a substituent is described as being “substituted” or “substitutable”, at least one H in the substituent is replaced with a non-hydrogen radical. Mono-substituted means having only one H replaced, di- or tri-substituted means having two or three hydrogens replaced, respectively, and so forth. If there are more than one substituents present, they can be identical or different. Thus, for example, 2-bromoethyl substituent —CH₂CH₂Br is regarded as a mono-substituted alkyl substituent, 3,5-dimethylphenyl or 2-chloro-4-isopropylnaphthyl are regarded as a di-substituted carboaryl substituents, and 1-phenyl-2,7-dimethyl-indolyl is regarded as a trisubstituted heteroaryl substituent.

If a substituent is described as being “optionally substituted”, the substituent may be either (I) not substituted or (2) substituted.

This specification uses the terms “substituent” and “radical” interchangeably.

It is understood that statements and graphic representations introduced using words “example”, “for example”, “for instance”, “exemplify”, “illustration”, “illustrate”, “such as” and the like are intended to illustrate, rather than limit the scope of, claims and definitions.

If substituents are described as being “independently selected” from a group, each substituent is selected independent of the other. Each substituent therefore may be identical to or different from the other substituent(s).

When a chemical formula is used to describe a substituent, the dash on the left side of the formula indicates the portion of the substituent that has the free valence.

When a chemical formula is used to describe a linking element between two other elements of a depicted chemical structure, the leftmost dash of the substituent indicates the portion of the substituent that is bound to the left element in the depicted structure. The rightmost dash, on the other hand, indicates the portion of the substituent that is bound to the right element in the depicted structure.

With reference to the use of the words “comprise” or “comprises” or “comprising” in this patent (including the claims), Applicants note that unless the context requires otherwise, those words are used on the basis of clear understanding that they are to be interpreted inclusively, rather than exclusively, and that Applicants intend each of those words to be so interpreted in construing this patent, including the claims below.

Materials and Characterization Methods

All reagents used for the preparation of the catalysts were obtained commercially and were used as received. Reactions involving higher than atmospheric pressures were carried out in 25 mL CajonD Airfreeg storage vessels available from Chemglass. The substrates used in the kinetic resolution experiments were either purchased or prepared according to literature procedures. Chloroform (OmniSolve grade, stabilized with nonpolar hydrocarbons) was used as received from EM Science. Deuterated chloroform was used as received from Cambridge Isotope Laboratories, Inc. Solvents used for chromatography were ACS or HPLC grade, as appropriate. Reactions were monitored by thin layer chromatography (TLC) using EM Science 60F silica gel plates. Flash column chromatography was performed over ICN Ecochrom silica gel (32-630m). Melting points were measured on a MeI-Temp 11 capillary melting point apparatus and are uncorrected. ¹H NMR and ¹³C NMR spectra were recorded on a Unity 300 MHz Varian spectrometer. The chemical shifts are reported as □ values (ppm) relative to TMS. High-Resolution mass spectral analyses were performed at Washington University MS Center on a Kratos MS-50TA spectrometer using the fast atom bombardment method (FAB). Methods used for kinetic resolution experiments, determination of ee's and calculation of conversions and selectivities were adopted from previously published work.^{3b,4a} HPLC analyses were performed using a Shimadzu LC system using isopropanol-hexanes mobile phase at a flow rate of 1 mL/min and UV detection at 254 nm. Specific optical rotations were measured using a Perkin-Elmer 241 polarimeter. The absolute chiralities of the products and the unreacted starting materials obtained by kinetic resolution were determined by the sign of the optical rotation of the unreacted starting materials.

### Examples of Preparation of Novel Catalysts

### Example 1

### Preparation of (R)-2-Phenyl-2,3-dihydroimidazo[1,2-a]pyridine

### Example 1a

### Preparation of (R)-N-(Pyridyl-2)-2-Hydroxy-1-phenylethylamine (1a)

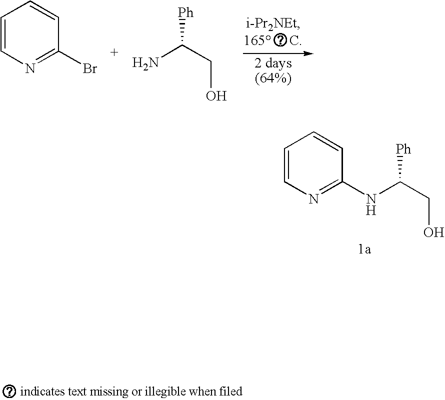

A 25-mL medium-pressure tube charged with 2-bromopyridine (158 mg, 1.00 mmol), (R)-phenylglycinol (137 mg, 1.00 mmol), N,N-diisopropylethylamine (150 mg, 1.16 mmol) and a stir bar was flushed with nitrogen several times, stoppered and heated at 160±5° C. for 2 days. The tube was allowed to cool to room temperature, the contents was diluted with a small amount of CH₂Cl₂ and chromatographed (hexanes-EtOAc 2:1→1:1→neat EtOAc) to afford 137 mg of the product, which crystallized quickly (64% yield). ¹H NMR (300 MHz, CDCl₃) □ 8.02 (dd; J=5.2 Hz, J₂=0.8 Hz; 1H), 7.26-7.40 (m, 6H), 6.57 (ddd; J=7.2 Hz, J₂=5.2 Hz, J₃=0.8 Hz; 1H), 6.29 (d, J=8.5 Hz; 1H), 5.51 (d, J=4.4 Hz, 1H), 5.28 (s, br, 1H), 4.78 (ddd, J=J₂=4.0 Hz, J₃ 8.0 Hz, 1H), 3.95 (m, 1H), 3.86 (dd; J=11.0 Hz, J₂=7.4 Hz; 1H); ¹³C NMR (75 MHz, CDCl₃) □ 158.7, 147.6, 140.5, 138.1, 129.1, 127.9, 127.0, 113.7, 108.3, 68.0, 59.6; MS: HR-FAB calculated for C₁₃H₁₄N₂O₂ (M+H⁺) m/z: 215.1184, measured m/z: 215.1181, error=−1.5 ppm; mp 104-106° C.; [□]_{D}=−21° (c=1.05, MeOH).

### Example 1b

### Preparation of R-2-Phenyl-2,3-dihydroimidazo[1,2-a]pyridine (2a)

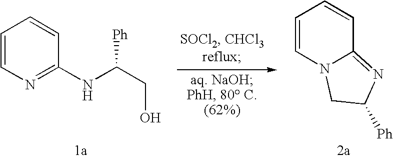

A solution of 1a (214 mg, 1.00 mmol) in 2 mL of CHCl₃ was treated with SOCl₂ (0.150 mL, 2.06 mmol) added dropwise at room temperature, then heated to reflux in an oil bath kept at 70° C. After 1 hour, the flask was taken out of the bath, allowed to cool and treated cautiously with 2-3 drops of MeOH (vigorous gas evolution!), then heated again for 5-10 minutes. The solvent was removed under reduced pressure, and the semicrystalline, gummy residue was extracted with warm water. The aqueous extract was filtered from gummy particles through a cotton plug, basified with concentrated NaOH and extracted with benzene (ca. 25 mL). The benzene solution was dried briefly over Na₂SO₄ and then heated under reflux in an oil bath at 80-85° C. After 2 hours, the reaction mixture was cooled to room temperature and extracted with water 3 times. The aqueous extract was made strongly basic with concentrated NaOH and extracted with CH₂Cl₂. The organic extract was dried over Na₂SO₄ and evaporated to give bright-yellow, crystalline product (121 mg, 62% yield). Recrystallization from boiling hexanes gave thin plates.

¹H NMR (300 MHz, CDCl₃) □ 7.24-7.35 (m, 5H), 6.90-6.93 (m, 1H), 6.82-6.88 (m, 1H), 6.42 (ddd, J₁=9.34 Hz, J₂=1.38 Hz, J₃=0.28 Hz, 1H), 5.66 (m, 1H), 5.23 (m, 1H), 4.36 (m, 1H), 3.84 (m, 1H); ¹³C NMR (75 MHz, CDCl₃) □ 158.4, 145.0, 137.0, 133.8, 128.8, 127.3, 126.8, 115.0, 103.2, 67.3, 57.6; MS: HR-FAB calculated for C₁₃HR₃N₂ (M+H⁺) m/z: 197.1079, measured m/z: 197.1070, error=−4.4 ppm; mp 101-103° C.; [DID=+412° (c=1.03, MeOH).

### Example 2

### Preparation of (R)-5-Bromo-2-phenyl-2,3-dihydromidazo[1,2-a]pyridine

### Example 2a

### Preparation of (R)-N-(5-Bromopyridyl-2)-2-Hydroxy-1-phenylethylamine (1b)

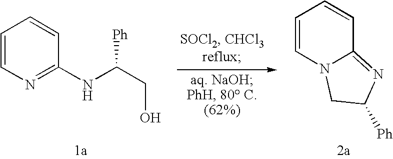

A solution of 1a (214 mg, 1.00 mmol) in 2 mL of CHCl₃ was treated with SOCl₂ (0.150 mL, 2.06 mmol) added dropwise at room temperature, then heated to reflux in an oil bath kept at 70° C. After 1 hour, the flask was taken out of the bath, allowed to cool and treated cautiously with 2-3 drops of MeOH (vigorous gas evolution!), then heated again for 5-10 minutes. The solvent was removed under reduced pressure, and the semicrystalline, gummy residue was extracted with warm water. The aqueous extract was filtered from gummy particles through a cotton plug, basified with concentrated NaOH and extracted with benzene (ca. 25 mL). The benzene solution was dried briefly over Na₂SO₄ and then heated under reflux in an oil bath at 80-85° C. After 2 hours, the reaction mixture was cooled to room temperature and extracted with water 3 times. The aqueous extract was made strongly basic with concentrated NaOH and extracted with CH₂Cl₂. The organic extract was dried over Na₂SO₄ and evaporated to give bright-yellow, crystalline product (121 mg, 62% yield). Recrystallization from boiling hexanes gave thin plates.

¹H NMR (300 MHz, CDCl₃) □ 7.24-7.35 (m, 5H), 6.90-6.93 (m, 1H), 6.82-6.88 (m, 1H), 6.42 (ddd, J₁=9.34 Hz, J₂=1.38 Hz, J₃=0.28 Hz, 1H), 5.66 (m, 1H), 5.23 (m, 1H), 4.36 (m, 1H), 3.84 (m, 1H); ¹³C NMR (75 MHz, CDCl₃) □ 158.4, 145.0, 137.0, 133.8, 128.8, 127.3, 126.8, 115.0, 103.2, 67.3, 57.6; MS: HR-FAB calculated for C₁₃H₁₃N₂ (M+H⁺) m/z: 197.1079, measured m/z: 197.1070, error=−4.4 ppm; mp 101-103° C.; [□]_{D}=+4120 (c=1.03, MeOH).

### Example 2b

### Preparation of (R)-5-Bromo-2-pyridyl-2,3-dihydroimidazo [1,2-a]pyridine (2b)

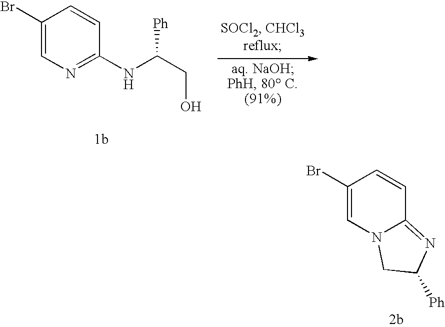

A procedure analogous to the cyclization of 1a was carried out on 0.5 mmol scale (147 mg of 1b, 0.075 mL SOCl₂, 1 mL CHCl₃) and produced 126 mg (91% yield) of yellow oil which crystallized quickly.

¹H NMR (300 MHz, CDCl₃) □ 7.26-7.37 (m, 5H), 7.07 (d, J=1.37 Hz, 1H), 6.86 (dd, J=9.89 Hz, J₂=2.20 Hz, 1H), 6.37 (dd, J=11.70 Hz, J₂=0.55 Hz, 1H), 5.25 (dd, J=11.54 Hz, 1H), 4.35 (t, J=10.98, 1H), 3.86 (dd, J₁=9.07 Hz, J₂=10.80 Hz, 1H); ¹³C NMR (75 MHz, CDCl₃) □ 156.5, 144.3, 140.0, 133.7, 129.0, 127.6, 126.8, 116.3, 94.3, 67.7, 57.8; MS: HR-FAB calculated for C₁₃H₁₂BrN₂ (M+H⁺) m/z: 275.0184, measured m/z: 275.0172, error=−4.3 ppm; mp 86-87.5° C.; [□]_{D}=+353° (c=1.03, MeOH).

### Example 3

### (R)-5-Nitro-2-phenyl-2,3-dihydroimidazo[1,2-a]pyridine

### Example 3a

### Preparation of 1c: (R)-N-(5-Nitropyridyl-2)-2-Hydroxy-1-phenylethylamine

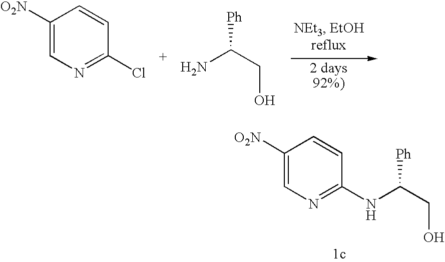

A solution of 2-chloro-5-nitropyridine (316 mg, 2.00 mmol), (R)-phenylglycinol (274 mg, 2.00 mmol) and NEt₃ (0.280 mL, 2.00 mmol) in 3.5 mL of absolute EtOH was refluxed under nitrogen for 2 days. EtOH was removed on a rotary evaporator and the residue was chromatographed (hexanes-EtOAc 3:1→1:1) to afford 480 mg of the product as orange, viscous oil, which crystallized after long standing (92% yield).

¹H NMR (300 MHz, CDCl₃) □ 8.96 (d, J=2.7 Hz, 1H), 8.12 (dd, J=9.1 Hz, J₂=2.7 Hz, 1H), 7.26-7.41 (m, 5H), 6.31 (d, J=9.1 Hz, 2H), 5.00 (s, br, 1H), 4.04 (dd, J=11.3 Hz, J₂=3.8 Hz, 1H), 3.95 (dd, J₁=11.3 Hz, J₂=5.8 Hz, 1H), 2.38 (s, br, 1H); ¹³C NMR (75 MHz, CDCl₃) □ 160.2, 145.9 (q, J=4.5 Hz), 139.5, 134.9 (q, J=1.5 Hz), 129.3, 128.3, 126.9, 124.6 (q, J=270 Hz), 116.4 (q, J=33.2 Hz), 107.4, 67.5, 58.8; MS: HR-FAB calculated for C₁₃H₁₃BrN₂O (M+H⁺) m/z: 260.1035, measured m/z: 260.1057, error=−4.4 ppm; mp109-110° C.; [□]_{D}=−165° (c=0.96, MeOH).

### Example 3b

### Preparation of (R)-5-Nitro-2-phenyl-2,3-dihydroimidazo[1,2-a]pyridine (2c)

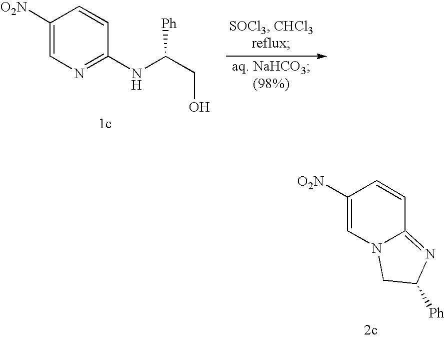

A solution of 1c (347 mg, 1.34 mmol) in 7 mL of CHCl₃ was treated with SOCl₂ (0.250 mL, 3.43 mmol) added dropwise at room temperature, then heated to reflux in an oil bath kept at 65° C. After 1.5 hours, the flask was taken out of the bath, allowed to cool somewhat and treated cautiously with 2-3 drops of MeOH (vigorous gas evolution!), then heated again for 5 minutes. The mixture was rotary evaporated and the evaporation residue was extracted with water. The aqueous extract was decanted from the gummy residue, brought to pH 7-8 with aqueous NaHCO₃ and extracted with CH₂Cl₂ 3 times. More aqueous NaHCO₃/NaOH was added to the aqueous phase to pH 12 and extraction was continued until organic extracts were pale-yellow (4 times). The organic phase was dried over NaOH pellets and then rotary evaporated. The crude mixture was chromatographed (20% i-PrOH+2% NEt₃ in hexanes) to give 318 mg of light-orange, non-crystalline mass (98%).

¹H NMR (300 MHz, CDCl₃) □ 8.38 (d, J=2.20 Hz, 1H), 7.59 (dd, J=10.44 Hz, J₂=7.97 Hz, 1H), 7.26-7.38 (m, 5H), 6.38 (d, J=10.44 Hz, 1H), 5.37 (dd, J₁=10.71 Hz, J₂=8.24 Hz, 1H), 4.46 (t, J=11.54, 1H), 3.94 (dd, J=11.82 Hz, J₂=8.24 Hz, 1H); ¹³C NMR (75 MHz, CDCl₃) □ 155.2, 142.5, 137.8, 130.9, 129.5, 129.1, 128.1, 126.7, 113.4, 68.8 57.1; MS: HR-FAB calculated for C₁₃H₁₂N₃O₂ (M+H⁺) m/z: 242.0930, measured m/z: 242.0930, error=0.2 ppm; [□]_{D}=+173° (c=0.94, MeOH).

### Example 4

### (R)-5-Trifluoromethyl-2-phenyl-2,3-dihydroimidazo[1,2-a]pyridine (CF₃-PIP)

### Example 4a

### Preparation of (R)-N-(5-Trifluoromethylpyridyl-2)-2-Hydroxy-1-phenylethylamine

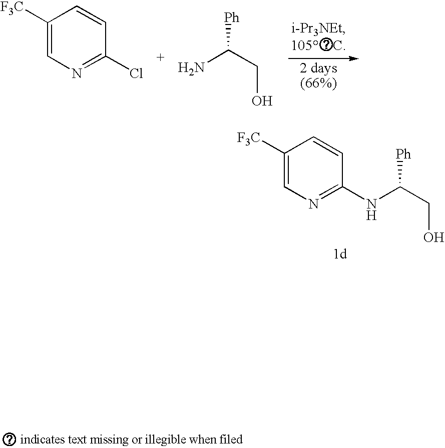

A 25 mL medium-pressure tube charged with 2-chloro-5-trifluoromethylpyridine (1.502 g, 8.270 mmol), (R)-phenylglycinol (1.102 g, 8.030 mmol), N,N-diisopropylethylamine (1.200 g, 9.300 mmol) and a stir bar was flushed with nitrogen several times, stoppered and heated at 105±5° C. for 2 days. The tube was allowed to cool to room temperature, the contents was diluted with a small amount of CH₂Cl₂ and chromatographed (isopropanol-hexanes 5% 10%) to afford 1.503 g of the product, which crystallized quickly (66% yield).

¹H NMR (300 MHz, CDCl₃) □ 8.28 (s, 1H), 7.52 (dd, J=8.79 Hz, J₂=2.20 Hz, 1H), 7.26-7.37 (m, 5H), 6.33 (d, J=8.79 Hz, 1H), 5.80 (d, J=5.50 Hz, 1H), 4.89 (dd, J=9.89 Hz, J₂=6.05 Hz, 1H), 3.99 (dd, J=11.26 Hz, J₂=3.84 Hz, 1H), 3.91 (dd, J=10.99 Hz, J₂=6.60 Hz, 1H), 3.28 (s, br, 1H); ¹³C NMR (75 MHz, CDCl₃) □ 161.0, 146.9, 138.7, 136.5, 133.4, 129.4, 128.5, 126.8, 106.7, 66.9, 58.3; MS: HR-FAB calculated for C₁₃H₁₃BrN₂O (M+H⁺) m/z: 283.1058, measured m/z: 283.1069, error=3.8 ppm; mp 105-105.5° C.; [□]_{D}=−57° (c=1.09, MeOH).

### Example 4b

### Preparation of (R)-5-Trifluoromethyl-2-phenyl-2,3-dihydroimidazo[1,2-a]pyridine (CF₃-PIP, 2d)

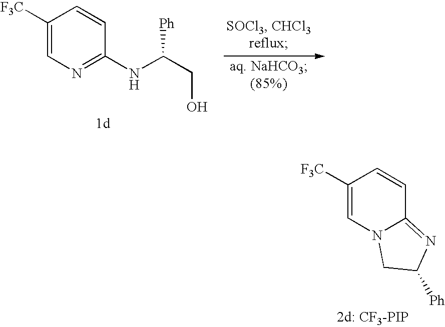

A procedure analogous to the cyclization of 1c was carried out on 5.24 mmol scale (1.499 g of 1d, 1.05 mL SOCl₂, 30 mL CHCl₃) and produced after chromatography (isopropanol-hexanes 5%+10%) 1.176 g (85% yield) of yellow oil which crystallized quickly. Recrystallization from hexanes produced yellow needles (78% recovery). Note: for the sake of consistency, all the data for kinetic resolution experiments shown in the table were obtained using the recrystallized material. However, the difference between it and the unrecrystallized product becomes appreciable only at selectivity levels of 25:1 and above.

¹H NMR (300 MHz, CDCl₃) □ 7.26-7.38 (m, 6H) 6.93 (dd, J₁=10.01 Hz, J₂=2.20 Hz, 1H) 6.46 (d, J=9.77, 1H) 5.29 (dd, J=11.48 Hz, J₂=8.79 Hz, 1H), 4.36 (t, J=11.23 Hz, 1H), 3.86 (t, J=9.74 Hz, 1H); ¹³C NMR (75 MHz, CDCl₃) □ 156.7, 143.8, 133.8 (q, J=5.54 Hz), 132.5 (q, J=2.51 Hz), 129.0, 127.8, 126.8, 124.0 (q, J=268 Hz), 115.6, 106.9 (q, J=34.8 Hz), 67.9, 57.2; MS: HR-FAB calculated for C₁₄H₁₂F₃LiN₂ (M+Li⁺) m/z: 271.1034, measured m/z: 271.1029, error=−1.9 ppm; mp 128-129° C. (from hexanes); [□]_{D}=+2770 (c=0.99, MeOH).

### Example 5

### (R)-7-Chloro-2-phenyl-2,3-dihydroimidazo[1,2-a]quinoline (CI-PIQ)

### Example 5a

### Preparation of (R)-N-(6-chloroquinolinyl-2)-2-Hydroxy-1-phenylethylamine (1e)

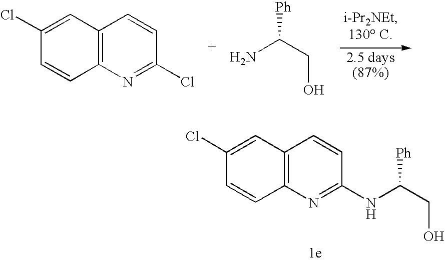

A 15-mL pressure tube charged with 2,6-dichloroquinoline (1.352 g, 6.83 mmol), (R)-phenylglycinol (0.964 g, 7.03 mmol), N,N-diisopropylethylamine (0.991 g, 7.67 mmol) and a stir bar was flushed with nitrogen several times, stoppered and heated at 130±5 C for 2.5 days. The tube was allowed to cool to room temperature and the content was diluted with CH₂Cl₂. DIPEAHCl precipitated out. The mixture was washed with saturated aqueous NH₄Cl to remove DIPEA and then with saturated aqueous NaHCO₃. The solution was dried over Na₂SO₄ and evaporated to afford 1.770 g of the crystalline product (87%), which was sufficiently pure for the next step. If necessary, the product can be chromatographed (6% isopropanol, 0.8% triethylamine in hexane).

¹H NMR (300 MHz, CDCl₃) □ 7.73 (d; J=9 Hz; 1H), 7.62 (d; J=8.7 Hz; 1H), 7.62 (d; J=8.7 Hz; 1H), 7.55 (d; J=2.2 Hz; 1H), 7.48 (dd; J₁=9 Hz, J₂=2.2 Hz; 1H), 7.28=7.45 (m; 5H), 6.67 (d; J=9 Hz; 1H), 5.85 (s; 1H), 5.16 (dt, J=7.4 Hz, J₂=3.6 Hz; 1H), 4.05 (dd; J=11.3 Hz, J₂=7.4 Hz; 1H), 3.00 (dd; J=11.3 Hz, J₂=3.6 Hz; 1H); ¹³C NMR (75 MHz, CDCl₃) □ 156.7, 144.8, 140.0, 137.0, 130.5, 129.0, 128.05, 127.98, 126.88, 126.81, 126.2, 123.9, 113.0, 68.3, 59.8.

### Example 5b

### Preparation of (R)-7-Chloro-2-phenyl-2,3-dihydroimidazo[1,2-a]quinoline (CI-PIQ, 2e)

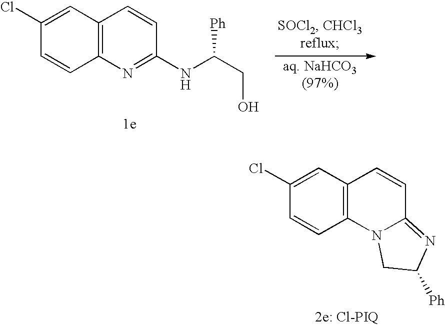

A solution of 1e (0.171 g, 0.57 mmol) in 3 mL of CHCl₃ was treated with SOCl₂ (0.10 mL, 1.37 mmol), and heated to reflux at 60-65 C. After 1.5 hours, the solution was allowed to cool, treated with drops of MeOH, and then heated again for 10 minutes. The solvent was removed under reduced pressure. The residue was dissolved in 5 mL of CH₂Cl₂; 3 mL of deionized water was added to the solution. The aqueous layer was basified to pH 8 with saturated aqueous NaHCO₃, then to pH 12 with aqueous NaOH (1M), and extracted with CH₂Cl₂. The organic extract was dried over Na₂SO₄, concentrated, and chromatographed (15% isopropanol, 2% triethylamine in hexanes) to afford 0.156 g of yellow oil (97%), which crystallized on standing.

¹H NMR (300 MHz, CDCl₃) □ 7.20-7.40 (m; 8H), 6.76 (d; J=9.3 Hz; 1H), 6.65 (d; J=8.8 Hz; 1H), 5.40 (dd; J₁=11.4 Hz, J₂=8.4 Hz; 1H), 4.40 (dd; J₁=11.4 Hz, J₂=10.3 Hz; 1H), 3.88 (dd; J₁=10.3 Hz, J₂=8.4 Hz; 1H); ¹³C NMR (75 MHz, CDCl₃) □ 156.2, 143.9, 137.5, 135.8, 130.2, 128.7, 127.5, 127.4, 126.6, 125.4, 122.2, 118.4, 112.7, 67.7, 54.1.

### Results

The detailed experimental procedures describing kinetic resolution of alcohols and determination of enantioselectivity are given below.

The following Examples are intended merely to be illustrative and not limiting in any way of reactions to prepare my novel DHIP derivative catalysts (chemical resolving agents) and to show the utility of the novel catalysts, catalytic compositions and methods. The catalysts of the invention are useful to catalyze a host of reactions and reaction types, some of which are those that are disclosed herein and which are therefore representative.

(C) The following kinetic resolution tests show the utility of the catalysts. All kinetic resolution experiments were carried out according to Procedures A, B, C, and D described below. Selectivities and conversions were determined as described in Procedure E. In all of the Examples given below, catalytic compounds of the invention synthesized according to the illustrations given above were used to produce enantiomeric excesses or to desymmetrize meso-diols (products). These products were recovered, isolated and characterized as set forth in detail below.

Procedure A: Variation of substituent X in DHIP catalysts (2a-d).

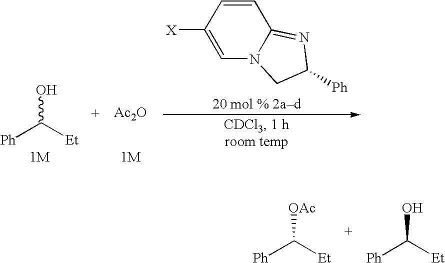

To a solution of 0.25 mmol of phenylethylcarbinol (34 □L, 34 mg) and 0.050 mmol of the catalyst (2a-d) in 0.250 mL CDCl₃ was added 0.25 mmol of acetic anhydride (24 □L, 26 mg). The mixture was swirled, left at room temperature for 1 hour, quenched by rapid addition of 0.25 mL of methanol, and left for one more hour. The reaction mixture was diluted with CH₂Cl₂, washed twice with 1 M HCl, then twice with saturated aqueous NaHCO₃, and dried over Na₂SO₄. The solution was concentrated on a rotary evaporator at room temperature and chromatographed (5-20% Et₂O in hexanes) to separate the ester from the unreacted alcohol.

Empirical test data illustrating effective kinetic resolutions brought about by using novel DHIP derivatives of the invention are presented in Table 1 below.

TABLE 1
Kinetic resolutions catalyzed by DHIP derivatives.

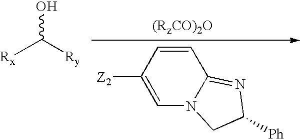

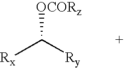

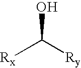

| | | | | | | | ee_{E} | Ee_{A} | C_{HPLC} | | C_{AVG} | |
|---|---|---|---|---|---|---|---|---|---|---|---|---|
| Entry | Rₓ | R_{y} | R_{z} | Z₂ | t(h) | # | % | % | % | s | % | s_{AVG} |
| 1^{a} | Phenyl | Et | Me | H | 1.0 | 1 | 49.0 | 12.8 | 20.7 | 3.3 | 21 | 3.3 |
| | | | | | | 2 | 49.5 | 13.4 | 21.1 | 3.36 | | |
| 2^{a} | ″ | Et | Me | Br | 1.0 | 1 | 74.4 | 25.2 | 25.3 | 8.7 | 25 | 8.6 |
| | | | | | | 2 | 74.0 | 24.4 | 24.8 | 8.5 | | |
| 3^{a} | ″ | Et | Me | NO₂ | 1.0 | 1 | 81.7 | 13.4 | 14.1 | 11.3 | 14 | 11 |
| | | | | | | 2 | 81.5 | 12.0 | 12.9 | 11.15 | | |
| 4^{a} | ″ | Et | Me | CF₃ | 1.0 | 1 | 79.5 | 51.9 | 39.5 | 14.6 | 38 | 14 |
| | | | | | | 2 | 79.4 | 46.3 | 36.8 | 13.7 | | |
| 5^{b} | ″ | Me | Me | CF₃ | 8 | 1 | 72.9 | 20.0 | 21.5 | 7.75 | 21 | 7.7 |
| | | | | | | 2 | 73.0 | 18.9 | 20.6 | 7.70 | | |
| 6^{b} | ″ | Et | Me | CF₃ | 8 | 1 | 80.7 | 60.6 | 42.9 | 17.2 | 43 | 17 |
| | | | | | | 2 | 81.0 | 59.6 | 42.4 | 17.4 | | |
| 7^{b} | ″ | i-Pr | Me | CF₃ | 30 | 1 | 82.4 | 79.2 | 49.0 | 24.8 | 47 | 24 |
| | | | | | | 2 | 82.9 | 70.3 | 45.9 | 22.4 | | |
| 8^{b} | ″ | Me | Et | CF₃ | 6 | 1 | 89.7 | 36.2 | 28.8 | 26.3 | 29 | 27 |
| | | | | | | 2 | 89.9 | 36.7 | 29.0 | 26.9 | | |
| 9^{b} | ″ | Et | Et | CF₃ | 6 | 1 | 90.3 | 64.3 | 41.6 | 38.3 | 42 | 38 |
| | | | | | | 2 | 90.2 | 64.7 | 41.8 | 37.8 | | |
| 10^{b} | ″ | i-Pr | Et | CF₃ | 30 | 1 | 80.9 | 97.6 | 54.7 | 41.1 | 55 | 41 |
| | | | | | | 2 | 78.7 | 98.9 | 55.7 | 41.8 | | |
| 11^{b} | ″ | i-Bu | Et | CF₃ | 52 | 1 | 93.5 | 88.2 | 48.6 | 87.1 | 48 | 85 |
| | | | | | | 2 | 93.8 | 83.6 | 47.1 | 82.5 | | |
| 12^{b} | 1-Naphthyl | Me | Et | CF₃ | 8 | 1 | 90.1 | 89.1 | 49.7 | 57.8 | 51 | 56 |
| | | | | | | 2 | 87.8 | 94.0 | 51.7 | 54.4 | | |
| 13^{b} | m-MeOC₆H₄ | Me | Et | CF₃ | 8 | 1 | 89.8 | 57.8 | 39.2 | 33.2 | 40 | 34 |
| | | | | | | 2 | 89.4 | 63.2 | 41.4 | 34.2 | | |
| 14^{b} | 2,4,6-Me₃C₆H₂ | Me | Et | CF₃ | 30 | 1 | 76.2 | 85.1 | 52.8 | 19.8 | 53 | 20 |
| | | | | | | 2 | 76.0 | 88.1 | 53.7 | 21.0 | | |

For each entry in the Table 1 above, two duplicate kinetic resolution tests were carried out, and thus the data shown in the last two columns (C_{AVG} and S_{AVG}) represent averages of two runs.

In the first schematic structure of Rx-OH-Ry, the Rx-OH-Ry represents a useful secondary alcohol substrate racemic composition which can be catalyzed by a novel catalyst of this discovery. Illustratively in that schematic structure Rx-OH-Ry, Rx and Ry are independently varying carbon atom containing moieties wherein Rx:A Ry and neither Rx nor Ry can be equal to H. Rx and Ry both can be independently selected from the following categories: substituted or unsubstituted aryl, including, but not limited to, phenyl and its derivatives; substituted or unsubstituted heteroaryl; substituted or unsubstituted aralkyl; substituted or unsubstituted bi-, tri- or polycyclic aromatic system, including, but not limited to, 1-naphthyl and 2-naphthyl, and also substituted or unsubstituted, branched or unbranched, linear and cyclic forms of the following: alkyl, alkenyl, and alkynyl. Also, cyclic structures arising from connecting R, and Ry are included herein as secondary alcohol substrates. Further, d,l- and meso-diols which under the reaction conditions would undergo double kinetic resolution or desymmetrization, respectively, are included herein. Specific structures shown in this specification are intended to exemplify, rather than delineate, the scope of suitable substrates.

The reaction time is expressed in hours and is shown under t(h). Entries 14 in Table 1 were obtained using 20 mol % of the catalyst in deuterated chloroform CDCl₃ at room temperature for 1 hour, and entries 5-14 were obtained using 2 mol % of the catalyst at 0° C. in the presence of N,N-diisopropylethylamine in chloroform CHCl₃ for the specified period of time. The esters produced and the unreacted alcohols resulting from kinetic resolution experiments were analyzed by chiral HPLC to determine their enantiomeric excesses (see below). HPLC analyses were performed on a Breeze LC system (Waters Corporation) and CHIRALCEL OD-H analytical column (4.6x250 mm, Chiral Technologies, Inc.) using isopropanol-hexanes mobile phase at a flow rate of 1 mL/min and UV detection at 254 nm. Specific optical rotations were measured using a Perkin-Elmer 241 polarimeter. The absolute chirality of the esters and alcohols obtained by kinetic resolution was determined by the sign of the optical rotation of the unreacted alcohol.

In an aspect of carrying out the catalytic reaction (kinetic resolution) of this discovery the secondary racemic alcohol is placed in contact with at least one of the novel catalysts. The catalyst and secondary racemic alcohol substrate are admixed together.

Generally the amount of catalyst used in such kinetic resolution of secondary racemic alcohols and desymmetrization of meso-diols is an amount which is effective to capably produce the desired kinetic resolution and desymmetrization. Thecatalyst contact time and temperature are such to capably provide sufficient kinetic resolution of the racemic secondary alcohol composition and desymmetrization of meso-diols so as to form desired products.

Further, kinetic resolution and desymmetrization are carried out for a time sufficient to produce a nonracemic composition comprising a nonracemic alcohol and an ester using an effective amount of at least one novel catalyst of this discovery. If desired, a nonracemic alcohol and ester product can be recovered from the composition for further use, and refined or purified as needed or desired.

C, in Table 1, is the “conversion”, which is defined as the ratio of the reaction product to the sum of the reaction product and the unreacted starting material, i.e., C=Prod/(Prod+SM) and can be calculated as C=eeA/(eeE+eeA); wherein eeA is the enantiomeric excess of the unreacted alcohol and eeE is the enantiomeric excess of the ester, wherein the term “enantiomeric excess” is defined as Ee=(M-m)/(M+m), wherein M is the amount of the major enantiomer and m is the amount of the minor enantiomer. The values of M and m are obtained by HPLC analysis.

TABLE 2

| Entry | X | t(h) | # | ee_{E} % | ee_{A} % | C_{HPLC} % | s | C_{AVG} % | s_{AVG} |
|---|---|---|---|---|---|---|---|---|---|
| 1 | H | 1.0 | 1 | 49.0 | 12.8 | 20.7 | 3.3 | 21 | 3.3 |
| | | | 2 | 49.5 | 13.4 | 21.1 | 3.36 | | |
| 2 | Br | 1.0 | 1 | 74.4 | 25.2 | 25.3 | 8.7 | 25 | 8.6 |
| | | | 2 | 74.0 | 24.4 | 24.8 | 8.5 | | |
| 3 | NO₂ | 1.0 | 1 | 81.7 | 13.4 | 14.1 | 11.3 | 14 | 11 |
| | | | 2 | 81.5 | 12.0 | 12.9 | 11.1 | | |
| 4 | CF₃ | 1.0 | 1 | 79.5 | 51.9 | 39.5 | 14.6 | 38 | 14 |
| | | | 2 | 79.4 | 46.3 | 36.8 | 13.7 | | |

Procedure B: Variation of the solvent

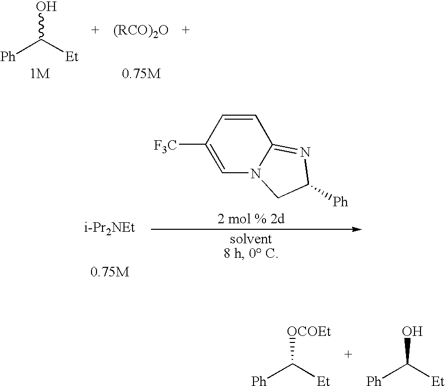

1) The stock solution of the catalyst was prepared by dissolving 0.040 mmol of 2d (10.6 mg) and 1.5 mmol of N,N-diisopropylethylamine (262]L, 194 mg) in the reaction solvent in a 2 mL volumetric test tube and bringing the volume to the mark.

2) A one-dram vial was charged with 0.5 mmol of (O)-phenyl ethyl carbinol and 0.500 mL of the stock solution of 2d cooled in an ice bath. After 15 minutes, 0.375 mmol of propionic anhydride was added. The mixture was swirled and left in the ice bath for 8 hours, at the end of which it was quenched by rapid addition of 0.5 mL of methanol, allowed to warm slowly and left for one more hour at room temperature. The workup and chromatography were carried out as described in Procedure A.

TABLE 3

| Entry | Solvent | # | ee_{E} % | ee_{A} % | C_{HPLC} % | s | C_{AVG} % | s_{AVG} |
|---|---|---|---|---|---|---|---|---|
| 1 | Chloroform | 1 | 90.4 | 60.8 | 40.2 | 36.8 | 39 | 36 |
| | | 2 | 90.6 | 57.2 | 38.7 | 36.0 | | |
| 2 | Diethyl ether | 1 | 93.5 | 35.8 | 27.7 | 42.0 | 27 | 40 |
| | | 2 | 92.9 | 34.6 | 27.1 | 38.3 | | |
| 3 | Toluene | 1 | 92.8 | 36.7 | 28.3 | 38.3 | 30 | 36 |
| | | 2 | 91.6 | 41.7 | 31.3 | 34.5 | | |
| 4 | Dichloromethane | 1 | 88.4 | 40.3 | 31.3 | 24.2 | 30 | 24 |
| | | 2 | 89.2 | 34.7 | 28.0 | 24.5 | | |
| 5 | t-Amyl alcohol | 1 | 90.4 | 21.4 | 19.2 | 24.3 | 18 | 23 |
| | | 2 | 89.9 | 18.7 | 17.2 | 22.5 | | |
| 6 | Acetonitrile | 1 | 81.5 | 19.5 | 19.3 | 11.8 | 20 | 11 |
| | | 2 | 79.3 | 19.6 | 19.8 | 10.5 | | |

Procedure C. Variation of the substrate and the acylating agent in the CF₃PIP-catalyzed kinetic resolutions.

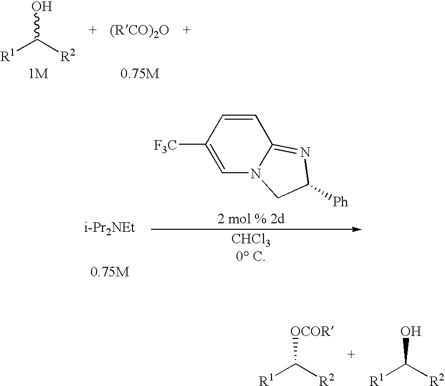

1) The stock solution of the catalyst was prepared by dissolving 0.100 mmol of 2d (26.4 mg) and 3.75 mmol of N,N-diisopropylethylamine (6540L, 485 mg) in CHCl₃ in a 5 mL volumetric flask and bringing the volume to the mark.

2) A one-dram vial was charged with 0.5 mmol of the racemic secondary alcohol and 0.500 mL of the stock solution of 2d, and cooled in an ice bath. After 15 minutes, 0.375 mmol of the anhydride was added. The mixture was swirled and left in the ice bath for a specified period of time, at the end of which it was quenched by rapid addition of 0.5 mL of methanol, allowed to warm slowly and left for one more hour at room temperature. The workup and chromatography were carried out as described in Procedure A.

TABLE 4

| Entry | R¹ | R² | R′ | t(h) | # | ee_{E} % | ee_{A} % | C_{HPLC} % | s | C_{AVG} % | s_{AVG} |
|---|---|---|---|---|---|---|---|---|---|---|---|
| 1 | Phenyl | Me | Me | 8 | 1 | 72.9 | 20.0 | 21.5 | 7.75 | 21 | 7.7 |
| | | | | | 2 | 73.0 | 18.9 | 20.6 | 7.70 | | |
| 2 | Phenyl | Et | Me | 8 | 1 | 80.7 | 60.6 | 42.9 | 17.2 | 43 | 17 |
| | | | | | 2 | 81.0 | 59.6 | 42.4 | 17.4 | | |
| 3 | Phenyl | i-Pr | Me | 30 | 1 | 82.4 | 79.2 | 49.0 | 24.8 | 47 | 24 |
| | | | | | 2 | 82.9 | 70.3 | 45.9 | 22.4 | | |
| 4 | Phenyl | Me | Et | 8 | 1 | 89.3 | 42.7 | 32.3 | 26.8 | 32 | 26 |
| | | | | | 2 | 89.2 | 40.3 | 31.1 | 26.0 | | |
| 5 | Phenyl | Et | Et | 8 | 1 | 90.4 | 60.8 | 40.2 | 36.8 | 39 | 36 |
| | | | | | 2 | 90.6 | 57.2 | 38.7 | 36.0 | | |
| 6 | Phenyl | i-Pr | Et | 30 | 1 | 80.9 | 97.6 | 54.7 | 41.1 | 55 | 41 |
| | | | | | 2 | 78.7 | 98.9 | 55.7 | 41.8 | | |
| 7 | Phenyl | t-Bu | Et | 52 | 1 | 93.5 | 88.2 | 48.6 | 87.1 | 48 | 85 |
| | | | | | 2 | 93.8 | 83.6 | 47.1 | 82.5 | | |
| 8 | 1-Naphthyl | Me | Et | 8 | 1 | 90.1 | 89.1 | 49.7 | 57.8 | 51 | 56 |
| | | | | | 2 | 87.8 | 94.0 | 51.7 | 54.4 | | |
| 9 | m-MeC₆H₄ | Me | Et | 8 | 1 | 88.1 | 51.7 | 37.0 | 26.3 | 36 | 27 |
| | | | | | 2 | 88.7 | 48.8 | 35.5 | 27.1 | | |
| 10 | m-MeOC₆H₄ | Me | Et | 8 | 1 | 89.8 | 57.8 | 39.2 | 33.2 | 40 | 34 |
| | | | | | 2 | 89.4 | 63.2 | 41.4 | 34.2 | | |
| 11 | m-BrC₆H₄ | Me | Et | 8 | 1 | 88.1 | 69.1 | 44.0 | 32.6 | 44 | 32 |
| | | | | | 2 | 87.8 | 69.7 | 44.2 | 32.0 | | |
| 12 | o-MeC₆H₄ | Me | Et | 8 | 1 | 86.4 | 66.3 | 43.4 | 27.3 | 44 | 26 |
| | | | | | 2 | 85.5 | 66.0 | 43.6 | 25.3 | | |
| 13 | 2,4,6- | Me | Et | 30 | 1 | 76.2 | 85.1 | 52.8 | 19.8 | 53 | 20 |
| | Me₃C₆H₂ | | | | 2 | 76.0 | 88.1 | 53.7 | 21.0 | | |
| 14 | Cyclohexyl | Me | Et | 50 | n/a | nd^{a} | nd^{a} | nd^{a} | nd^{a} | <4^{b} | nd^{a} |

| | | | | | | | | | | |
|---|---|---|---|---|---|---|---|---|---|---|
| 15 | 1-Indanol | Et | 50 | n/a | ≈0 | ≈0 | nd^{a} | ≈1 | 16^{b} | ≈1 |

^{a}Not determined
^{b}Determined by ¹H NMR

Procedure D. Preparative scale resolution of (O)-1-(1-naphthyl)-1-ethanol using CF₃PIP.

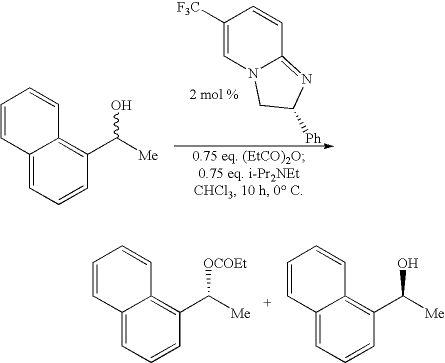

The same proportions were used as in Procedure B described above. A solution of the substrate (2.416 g, 14.0 mmol), DIPEA (1.93 mL, 10.5 mmol) and CF₃—PIP 2d (74 mg, 0.28 mmol) in 14 mL of chloroform was stirred magnetically in an ice bath for 15 minutes, then treated with propionic anhydride (1.35 mL, 10.5 mmol). The mixture was stirred at 0° C. for 10 hours, at which time it was quenched with methanol (10 mL), allowed to warm slowly and left for one more hour at room temperature. The reaction mixture was diluted with CH₂Cl₂, washed twice with 1 M HCl, then twice with saturated aqueous NaHCO₃, and dried over Na₂SO₄. The solution was concentrated on a rotary evaporator at room temperature and chromatographed (5-20% Et₂O in hexanes). The ester was eluted first (1.672 g, 7.32 mmol, 52% yield), followed by the unreacted alcohol (1.091 g, 6.33 mmol, 45% yield). The enantiomeric excess of the ester was determined by HPLC (vide infra) to be 82.5%, and that of the alcohol, 98.8%. Based on these ee values, the conversion was calculated to be 54.5% (cf 53.6% conversion based on the isolated materials), and the selectivity factor, 52.3. The aqueous phase obtained during the workup was basified with 0.5 M NaOH and extracted with CH₂Cl₂ several times until the aqueous phase was pale-yellow. The extract was dried over Na₂SO₄, evaporated, and chromatographed to give 50 mg of pure CF₃—PIP (68% recovery).

Procedure E. Comparison of CF₃—PIP- and Cl-PIQ-catalyzed kinetic resolutions of alcohols.

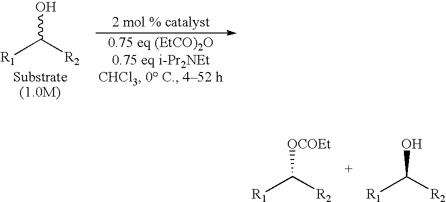

1) Stock solutions of catalysts CF₃—PIP 2d and Cl-PIQ 2e were prepared as described above in Procedure C.

2) CF₃PIP-catalyzed kinetic resolutions were carried out as described above in Procedure C using propionic anhydride. Cl-PIQ-catalyzed kinetic resolutions were carried out under identical conditions, except Entries 12 and 16, which required shorter times than with CF₃PIP to reach useful levels of conversion.

TABLE 5

| | | | | | ee_{E} | ee_{A} | C_{HPLC} | | C_{AVG} | |
|---|---|---|---|---|---|---|---|---|---|---|
| Entry | Substrate | Catalyst | t(h) | # | % | % | % | s | % | s_{AVG} |
| 1 2 | 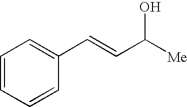 | CF₃PIP Cl-PIQ | 8 8 | 1 2 1 2 | 79.1 82.2 86.3 85.7 | 11.8 13.6 66.1 68.7 | 13.0 14.2 43.4 44.5 | 9.6 11.7 26.9 26.7 | 14 44 | 11 |
| # 27 | | | | | | | | | | |
| 3 4 | 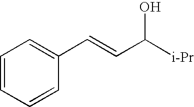 | CF₃PIP Cl-PIQ | 8 8 | 1 2 1 2 | 86.9 86.9 75.9 81.3 | 39.2 36.4 90.5 84.8 | 31.1 29.5 54.4 51.1 | 21.0 20.4 22.3 25.9 | 30 53 | 21 |
| # 24 | | | | | | | | | | |
| 5 6 | 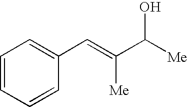 | CF₃PIP Cl-PIQ | 8 8 | 1 2 1 2 | 75.2 80.9 82.1 82.0 | 12.4 13.4 53.5 45.7 | 14.1 14.2 39.4 35.8 | 8.0 10.8 17.3 15.8 | 14 37.6 | 9 |
| # 16.6 | | | | | | | | | | |
| 7 8 | 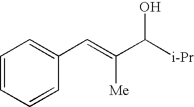 | CF₃PIP Cl-PIQ | 8 8 | 1 2 1 2 | 84.1 84.0 86.6 88.2 | 8.7 8.4 42.3 39.7 | 9.4 9.1 32.8 31.0 | 12.6 12.5 21.1 23.6 | 9 32 | 13 |
| # 22 | | | | | | | | | | |
| 9 10 | 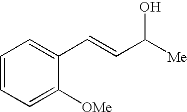 | CF₃PIP Cl-PIQ | 8 8 | 1 2 1 2 | 61.6 71.6 75.7 77.8 | 23.9 24.2 96.3 97.2 | 27.9 25.3 56.0 55.6 | 5.3 7.6 28.1 33.3 | 27 56 | 6 |
| # 31 | | | | | | | | | | |
| 11 12 | 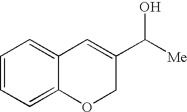 | CF₃PIP Cl-PIQ | 8 8 | 1 2 1 2 | 81.9 82.1 80.6 80.7 | 83.0 82.4 99.7 99.6 | 50.3 50.1 55.3 55.3 | 25.9 25.8 58.2 56.0 | 50 55 | 26 |
| # 57 | | | | | | | | | | |
| 13 14 | 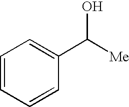 | CF₃PIP Cl-PIQ | 8 8 | 1 2 1 2 | 89.3 89.2 78.3 78.6 | 42.7 40.3 96.5 96.2 | 32.3 31.1 55.2 55.1 | 26.8 26.0 32.6 32.5 | 32 55 | 26 |
| # 33 | | | | | | | | | | |
| 15 16 | 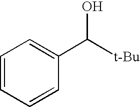 | CF₃PIP Cl-PIQ | 52 8 | 1 2 1 2 | 93.5 93.8 96.7 96.1 | 88.2 83.6 69.7 72.5 | 48.6 47.1 41.9 43.0 | 87.1 82.5 124 110 | 48 42 | 85 |
| # 117 | | | | | | | | | | |

Procedure F. Kinetic resolutions of oxazolidinones using CF₃—PIP and Cl-PIQ.

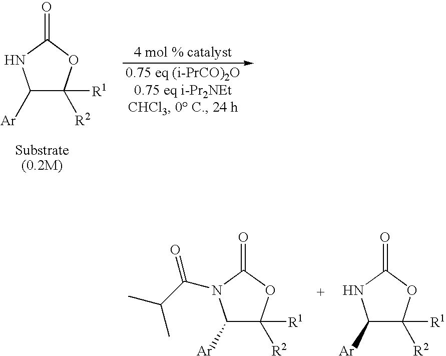

1) The stock solutions of the catalysts was prepared by dissolving 0.04 mmol of CF₃PIP 2d (10.6 mg) or 0.04 mmol of Cl-PIQ 2e (11.2 mg), respectively, and 0.75 mmol of N,N-diisopropylethylamine (131 □L, 97 mg) in CHCl₃ in a 5 mL volumetric flask and bringing the volume to the mark.

2) Racemic oxazolidinone (0.20 mmol) was placed in a 1 dram vial. To this was added 1 mL of the catalyst solution. In a few cases it was necessary to carefully warm the vial to effect complete solution of the substrate. The solutions were then cooled to 0° C., 0.15 mmol of isobutyric anhydride (24 mg) was added and the reaction mixture was kept at 0° C. After 24 h, it was quenched by addition of 1 mL of MeOH at 0° C., allowed to stand for 30 min and then diluted to approximately 7 mL with CH₂Cl₂. The solution was washed once with 1N HCl, dried over Na₂SO₄, concentrated, and the substrate/product mixtures purified by flash chromatography (35% ethyl acetate in hexane). The fractions were concentrated down by a continuous air stream and those fractions containing substrate and product were collected and analyzed by chiral HPLC. Duplicate results were obtained for each kinetic resolution experiment.

TABLE 6

| | | | | | ee_{E} | ee_{SM} | C_{HPLC} | | C_{AVG} | |
|---|---|---|---|---|---|---|---|---|---|---|
| Entry | Substrate | Catalyst | t(h) | # | % | % | % | s | % | s_{AVG} |
| 1 2 | 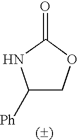 | CF₃PIP Cl-PIQ | 24 24 | 1 2 1 2 | 84.9 85.2 86.3 88.6 | 42.5 40.6 68.8 67.1 | 33.3 32.3 44.4 43.1 | 18.6 18.5 27.9 33.3 | 33 44 | 19 |
| # 31 | | | | | | | | | | |
| 3 4 | 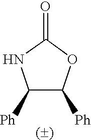 | CF₃PIP Cl-PIQ | 24 24 | 1 2 1 2 | 84.0 80.2 84.1 84.2 | 60.5 61.5 81.6 66.9 | 41.9 43.4 49.2 44.3 | 21.2 17.0 29.1 23.3 | 43 47 | 19 |
| # 26 | | | | | | | | | | |
| 5 6 | 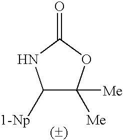 | CF₃PIP Cl-PIQ | 24 24 | 1 2 1 2 | 92.3 93.2 95.4 94.5 | 34.2 47.4 52.8 58.3 | 26.9 33.7 35.6 38.2 | 38.1 45.7 71.8 63.9 | 30 37 | 42 |
| # 68 | | | | | | | | | | |

Procedure G. Determination of Enantiomeric Excesses, Conversions and Selectivities in kinetic resolutions of alcohols and oxazolidinones.

All the alcohols recovered from kinetic resolutions using the R-enantiomers of the catalysts 2a-e were found to be enriched in the S-enantiomer according to the negative sign of optical rotation.

The unreacted oxazolidinones recovered from kinetic resolutions using the R-enantiomers of the catalysts 2d and 2e were found to be enriched in the R-enantiomer (configurationally analogous to S-alcohols), as determined by comparison of their optical rotations with those described in the literature. When the S-enantiomer of 2e was used, the S-enantiomers of the oxazolidinones were recovered.

The enantiomeric excesses of the alcohols were determined by HPLC using a CHIRALCEL OD-H analytical column (4.6x250 mm, Chiral Technologies, Inc.) and isopropanouhexanes mixtures as mobile phase. The enantiomeric excesses of the esters were determined, in most cases, by hydrolysis to the parent alcohols (2 mL of 2 M KOH in methanol, at room temperature until complete by TLC), which were analyzed as described above. 1-(1-naphthyl)-1-ethyl propionate was analyzed directly on CHIRALCEL OD-H analytical column using 3% isopropanol in hexanes.

The enantiomeric excesses of the oxazolidinones and their N-isobutyryl derivatives were determined using a CHIRALPAK AD analytical column (4.6x250 mm, Chiral Technologies, Inc.) and isopropanoluhexanes mixtures as mobile phase.

The enantioselectivity s was calculated according to the equation:
s=ln((1−C)(1−ee_{SM})/ln((1−C)(1+ee_{SM})),

The conversion C used in the above equation was calculated as C=ee_{SM}/(ee_{P}+ee_{SM}), where ee_{P} is the enantiomeric excess of the product and ee_{SM} is the enantiomeric excess of the unreated starting material.

### ADDITIONAL EXAMPLES OF CATALYTIC SPECIES OF THE INVENTION

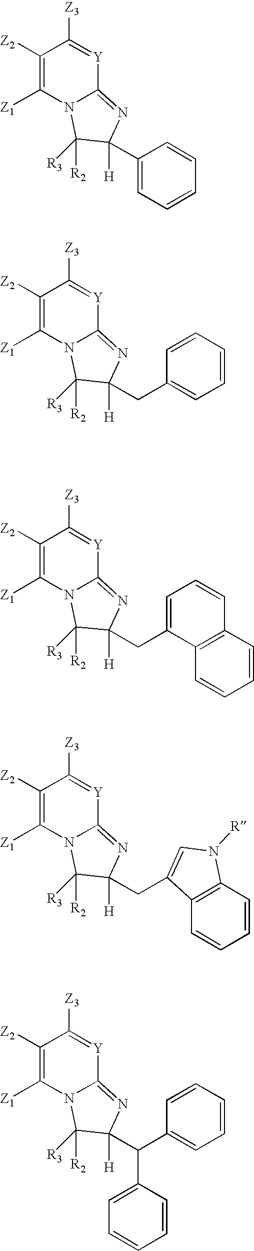 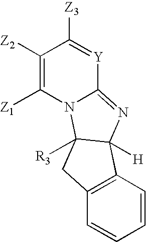 [0180] wherein R2 and R3 can be varied independently and can be defined as any of the following:
hydrogen, substituted or unsubstituted alkyl, substituted or unsubstituted aralkyl, substituted or unsubstituted alkenyl, substituted or unsubstituted alkynyl, substituted or unsubstituted aryl, substituted or unsubstituted heteroaryl;
carbonyl derivative, as defined below and
wherein R₁ and R₂ can jointly form cyclic structures, including, but not limited to, indane derivatives such as R₁; or R₂ and R₃ can jointly form cyclic structures and
wherein R₁, R₂, and R₃ each represent independently variable substituted or unsubstituted alkyl, substituted or unsubstituted aralkyl, substituted or unsubstituted alkenyl, substituted or unsubstituted alkynyl, substituted or unsubstituted aryl, substituted or unsubstituted heteroaryl; and wherein cyclic structures arising from co nnecting R₁ and R₂, or R₂ and R₃ are likewise included;

[image]

9) sulfenyl, sulfinyl and sulfonyl derivatives including, but not limited to, the classes described in FIG. 4, wherein R represents substituted or unsubstituted alkyl, substituted or unsubstituted aralkyl, substituted or unsubstituted alkenyl, substituted or unsubstituted alkynyl, substituted or unsubstituted aryl, substituted or unsubstituted heteroaryl;
wherein R₁, R₂, and R₃ each represent independently variable substituted or unsubstituted alkyl, substituted or unsubstituted aralkyl, substituted or unsubstituted aryl, substituted or unsubstituted heteroaryl; and wherein cyclic structures arising from connecting Rland R₂, or R₂ and R₃ are likewise included;

[image]

10) phosphoryl derivatives including, but not limited to, the classes described in FIG. 4 wherein R₁ and R₂ each represent independently variable substituted or unsubstituted alkyl, substituted or unsubstituted aralkyl, substituted or unsubstituted alkenyl, substituted or unsubstituted alkynyl, substituted or unsubstituted aryl, substituted or unsubstituted heteroaryl;
wherein R₃, R₄, and R₅ each represent independently variable substituted or unsubstituted alkyl, substituted or unsubstituted aralkyl, substituted or unsubstituted alkenyl, substituted or unsubstituted alkynyl, substituted or unsubstituted aryl, substituted or unsubstituted heteroaryl; and wherein cyclic structures arising from connecting R₁, R₂, R₃, R₄, R₅ and R₆ in any combination are likewise included;

[image]

[image]

11) arylazo group, i.e. N═N—R where R=substituted or unsubstituted aryl, substituted or unsubstituted heteroaryl;

12) amino group derivatives, including, but not limited to, the classes described in FIG. 2, wherein R₁ and R₂, R₃ and R₄ each represent independently variable substituted or unsubstituted alkyl, substituted or unsubstituted aralkyl, substituted or unsubstituted alkenyl, substituted or unsubstituted alkynyl, substituted or unsubstituted aryl, substituted or unsubstituted heteroaryl; and wherein cyclic structures arising from connecting R₁, R₂, R₃ and R₄ in any combination are likewise included;

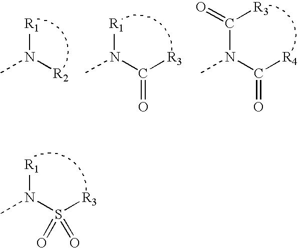

13) hydroxy group derivatives including, but not limited to, the classes described in FIG. 2 wherein R₁, R₂ and R₃ each represent independently variable substituted or unsubstituted alkyl, substituted or unsubstituted aralkyl, substituted or unsubstituted alkenyl, substituted or unsubstituted alkynyl, substituted or unsubstituted aryl, substituted or unsubstituted heteroaryl; and wherein cyclic structures arising from connecting, R₂ and R₃ are likewise included.

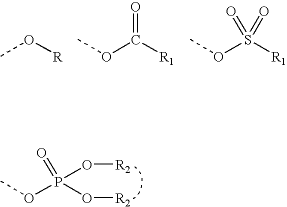

### ADDITIONAL EXAMPLES

A catalyzable composition comprises at least one asymmetric nucleophilic catalyst having a structure selected from the group consisting of consisting of (R)-N-(Pyridyl-2)-2-Hydroxy-1-phenylkethylanine, (R)-2-Phenyl-2,3-dihydroimadidazo [1,2-a]pyridine, (R)-N-(5-Bromopyridyl-2)-2-Hydroxy-1-phenylethylamine, (R)-5-Bromo-2-phenyl-2,3-dihydroimidazo[1,2-a]pyridine, (R)-N-(5-Nitro pyridyl-2)-2-Hydroxy-1-phenylethylanine, (R)-5-Nitro-2-Phenyl-2,3-dihydroimidazo{1,2-a]pyridine, (R)-N-(5-Trithoromethylpyridyl-2)-2-Hydroxy-1-Phenylethlamine and (R)-5-Trifluoromethyl-2-Phenyl-2,3-dihydromidazo[1,2-a]pyridine and a racemic alcohol or meso-diol composition

A method for preparing a nonracemic alcohol composition or a desymmetrized meso-diol composition comprises forming a composition comprising at least one asymmetric nucleophilic catalyst having a structure selected from the group consisting of consisting of (R)-N-(Pyridyl-2)-2-Hydroxy-1-phenylkethylanine, (R)-2-Phenyl-2,3-dihydroimadidazo[1,2-a]pyridine, (R)-N-(5-Bromopyridyl-2)-2-Hydroxy-1-phenylethylamine, (R)-5-Bromo-2-phenyl-2,3-dihydroimidazo[1,2-a]pyridine, (R)-N-(5-Nitro pyridyl-2)-2-Hydroxy-1-phenylethylanine, (R)-5-Nitro-2-Phenyl-2,3-dihydroimidazo{1,2-a]pyridine, (R)-N-(5-Trithoromethylpyridyl-2)-2-Hydroxy-1-Phenylethlamine and (R)-5-Trifluoromethyl-2-Phenyl-2,3-dihydromidazo[1,2-a]pyridine and at least one of a racemic alcohol and meso-diol composition and subjecting the composition to suitable time and temperature parameters to form a nonracemic alcohol or desymmetrized meso-diol composition. In an aspect a product is recovered from the catalyzed composition.

A method for kinetically resolving a nonracemic secondary alcohol composition comprises forming a catalyzable composition comprising at least one asymmetric nucleophilic catalyst having a structure of at least one structure from among the structures having a structure selected from the group consisting of consisting of (R)-N-(Pyridyl-2)-2-Hydroxy-1-phenylkethylanine, (R)-2-Phenyl-2,3-dihydroimadidazo[1,2-a]pyridine, (R)-N-(5-Bromopyridyl-2)-2-Hydroxy-1-phenylethylamine, (R)-5-Bromo-2-phenyl-2,3-dihydroimidazo[1,2-a]pyridine, (R)-N-(5-Nitro pyridyl-2)-2-Hydroxy-1-phenylethylanine, (R)-5-Nitro-2-Phenyl-2,3-dihydroimidazo{1,2-a]pyridine, (R)-N-(5-Trithoromethylpyridyl-2)-2-Hydroxy-1-Phenylethlamine and (R)-5-Trifluoromethyl-2-Phenyl-2,3-dihydromidazo[1,2-a]pyridine and a racemic alcohol or meso-diol composition and a racemic secondary alcohol composition and subjecting the catalyzable composition to catalytically suitable time and temperature parameters to form a nonracemic secondary alcohol composition and desymmetrized meso-diols.

A method for desymmetrizing a composition comprises forming a catalyzable composition comprising at least one asymmetric nucleophilic catalyst having a structure of at least one structure from among having a structure selected from the group consisting of consisting of (R)-N-(Pyridyl-2)-2-Hydroxy-1-phenylkethylanine, (R)-2-Phenyl-2,3-dihydroimadidazo[1,2-a]pyridine, (R)-N-(5-Bromopyridyl-2)-2-Hydroxy-1-phenylethylamine, (R)-5-Bromo-2-phenyl-2,3-dihydroimidazo[1,2-a]pyridine, (R)-N-(5-Nitro pyridyl-2)-2-Hydroxy-1-phenylethylanine, (R)-5-Nitro-2-Phenyl-2,3-dihydroimidazo{1,2-a]pyridine, (R)-N-(5-Trithoromethylpyridyl-2)-2-Hydroxy-1-Phenylethlamine and (R)-5-Trifluoromethyl-2-Phenyl-2,3-dihydromidazo[1,2-a]pyridine and a racemic alcohol or meso-diol composition and a meso-diol and subjecting the catalyzable composition to catalytically suitable time and temperature parameters to form a desymmetrized meso-diol.

While the invention has been disclosed by reference to the details of the preferred embodiments of the invention, it is to be understood that the disclosure is intended in an illustrative rather than in a limiting sense, as it is contemplated that modifications will readily occur to those skilled in the art, within the spirit of the invention and the scope of the appended claims.

1. A compound in racemic or non-racemic form, or salt thereof, wherein the compound has a structure represented by general formula 1: 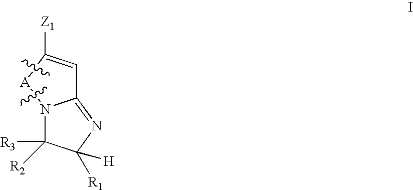
wherein A is selected from the group consisting of: 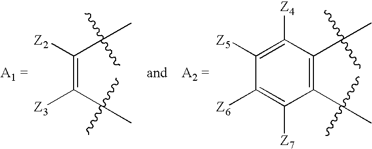
wherein R₁≠H and R₂ and R₃ can be H, and in addition R₁, R₂ and R₃ each are independently selected from the group consisting of alkyl, alkenyl, alkynyl, carboaryl, heteroaryl, carbocyclyl, heterocyclyl, oxycarbonyl, aminocarbonyl, each of which can optionally be substituted with one or more substituents, each independently selected from the group consisiting of (i) unsubstitutable substituents: halogen, cyano, nitro, oxo, and (ii) substitutable substituents: acyl, oxycarbonyl, aminocarbonyl, alkylsulfonyl, arylsulfonyl, aminosulfonyl, hydroxy, acyloxy, alkoxy, aralkoxy, alkylthio, arylthio, aralkylthio, mono- and dialkylamino, mono- and diaralkylamino, cycloamino, carboxamido, sulfamido, trialkylsilyl, alkyl, alkenyl, alkynyl, carboaryl, heteroaryl, carbocyclyl, heterocyclyl; wherein each of the substitutable subsituents, in turn, can optionally be substituted with one or more substituents independently selected from the group consisiting of halogen, cyano, nitro, oxo, acyl, oxycarbonyl, aminocarbonyl, alkylsulfonyl, arylsulfonyl, aminosulfonyl, hydroxy, acyloxy, alkoxy, aralkoxy, alkylthio, arylthio, aralkylthio, mono- and dialkylamino, mono- and diaralkylamino, cycloamino, carboxamido, sulfamido, trialkylsilyl, alkyl, alkenyl, alkynyl, carboaryl, heteroaryl, carbocyclyl, heterocyclyl;
wherein R₁ and R₂, and/or R₁ and R₃, and/or R₂ and R₃, can optionally form cyclic structures containing 5 to 10 members wherein any member of the cyclic structure is optionally substituted with one or more substituents independently selected from the group consisiting of (i) unsubstitutable substituents: halogen, cyano, nitro, oxo, and (ii) substitutable substituents: acyl, oxycarbonyl, aminocarbonyl, alkylsulfonyl, arylsulfonyl, aminosulfonyl, hydroxy, acyloxy, alkoxy, aralkoxy, alkylthio, arylthio, aralkylthio, mono- and dialkylamino, mono- and diaralkylamino, cycloamino, carboxamido, sulfamido, trialkylsilyl, alkyl, alkenyl, alkynyl, carboaryl, heteroaryl, carbocyclyl, heterocyclyl; each substitutable substituent of which, in turn, can optionally be substituted with one or more substituents independently selected from the group consisiting of halogen, cyano, nitro, oxo, acyl, oxycarbonyl, aminocarbonyl, alkylsulfonyl, arylsulfonyl, aminosulfonyl, hydroxy, acyloxy, alkoxy, aralkoxy, alkylthio, arylthio, aralkylthio, mono- and dialkylamino, mono- and diaralkylamino, cycloamino, carboxamido, sulfamido, trialkylsilyl, alkyl, alkenyl, alkynyl, carboaryl, heteroaryl, carbocyclyl, heterocyclyl;
wherein Z₁, Z₂, Z₃ Z₄, Z₅, Z₆ and Z₇ are each independently selected from the group consisting of (i) unsubstitutable substituents: halogen, cyano, nitro, arylazo, oxo, and (ii) substitutable substituents: acyl, oxycarbonyl, aminocarbonyl, alkylsulfonyl, arylsulfonyl, aminosulfonyl, hydroxy, acyloxy, alkoxy, aralkoxy, alkylthio, arylthio, aralkylthio, mono- and dialkylamino, mono- and diaralkylamino, cycloamino, carboxamrido, sulfamido, trialkylsilyl, alkyl, alkenyl, alkynyl, carboaryl, heteroaryl, carbocyclyl, heterocyclyl; each substitutable substituent of which can optionally be substituted with one or more substituents independently selected from the group consisiting of halogen, cyano, nitro, arylazo, oxo, acyl, oxycarbonyl, aminocarbonyl, alkylsulfonyl, arylsulfonyl, aminosulfonyl, hydroxy, acyloxy, alkoxy, aralkoxy, alkylthio, arylthio, aralkylthio, mono- and dialkylamino, mono- and diaralkylamino, cycloamino, carboxamido, sulfamido, trialkylsilyl, alkyl, alkenyl, alkynyl, carboaryl, heteroaryl, carbocyclyl, heterocyclyl; wherein each substitutable substituent of which, in turn, can optionally be substituted with one or more substituents independently selected from the group consisiting of halogen, cyano, nitro, arylazo, oxo, acyl, oxycarbonyl, aminocarbonyl, alkylsulfonyl, arylsulfonyl, aminosulfonyl, hydroxy, acyloxy, alkoxy, aralkoxy, alkylthio, arylthio, aralkylthio, mono- and dialkylamino, mono- and diaralkylamino, cycloamino, carboxamido, sulfamido, trialkylsilyl, alkyl, alkenyl, alkynyl, carboaryl, heteroaryl, carbocyclyl, heterocyclyl;
wherein Z₁ and Z₂ and/or Z₂ and Z₃ and/or Z₁ and Z₄ and/or Z₄ and Z₅ and/or Z₅ and Z₆ and/or Z₆ and Z₇ can optionally form cyclic structures containing 5 to 10 members wherein any member of the cyclic structure can optionally be substituted with one or more substituents independently selected from the group consisiting of halogen, cyano, nitro, arylazo, oxo, acyl, oxycarbonyl, aminocarbonyl, alkylsulfonyl, arylsulfonyl, aminosulfonyl, hydroxy, acyloxy, alkoxy, aralkoxy, alkylthio, arylthio, aralkylthio, mono- and dialkylamino, mono- and diaralkylamino, cycloamino, carboxamido, sulfamido, trialkylsilyl, alkyl, alkenyl, alkynyl, carboaryl, heteroaryl, carbocyclyl, heterocyclyl;
provided that when the compound is present in racemic form, and A=A₂ and Z₄, Z₅, Z₆, Z₇, R₂ and R₃ are all H, then R₁ cannot be phenyl, 4-fluorophenyl, 4-chlorophenyl, or 2-naphthyl.

2. A compound or salt thereof according to claim 1 wherein A=A₁ and R₃ is H and thus the compound has a structure represented by general formula II: 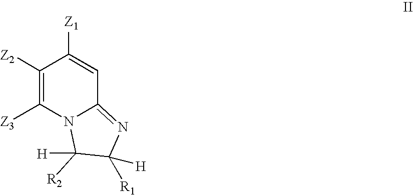
wherein R₁, R₂, Z₁, Z₂, and Z₃ are as defined in claim 1.

3. A compound or salt thereof according to claim 2 wherein:
Z₁, Z₃, and R₂ are all H; and
R₁ is selected from the group consisting of branched or unbranched alkyl, cycloalkyl, carboaryl and heteroaryl, each of which can optionally be substituted with one or more substituents independently selected from the group consisiting of halogen, cyano, nitro, acyl, oxycarbonyl, aminocarbonyl, alkylsulfonyl, arylsulfonyl, aminosulfonyl, hydroxy, acyloxy, alkoxy, aralkoxy, alkylthio, arylthio, aralkylthio, dialkylamino, cycloamino, carboxamido, sulfamido, trialkylsilyl, alkyl, aralkyl, perhaloalkyl, alkenyl, alkynyl, carboaryl, heteroaryl, carbocyclyl, heterocyclyl; and
Z₂ is selected from the group consisting of hydrogen, halogen, cyano, nitro, acyl, oxycarbonyl, aminocarbonyl, alkylsulfonyl, arylsulfonyl, aminosulfonyl, carboxamido, sulfamido, perhaloalkyl, carboaryl, heteroaryl, carbocyclyl, heterocyclyl.

4. A compound or salt thereof according to claim 3 wherein:
R₁ is a phenyl group optionally substituted with between 1 and 5 substituents independently selected from the group consisiting of halogen, cyano, nitro, acyl, alkoxycarbonyl, mono- and dialkylaminocarbonyl, alkylsulfonyl, arylsulfonyl, mono- and dialkylaminosulfonyl, hydroxy, acyloxy, alkoxy, aralkoxy, alkylthio, arylthio, aralkylthio, dialkylamino, cycloamino, carboxamido, sulfamido, trialkylsilyl, alkyl, aralkyl, trifluoromethyl, carboaryl, and heteroaryl; and
Z₂ is as defined in claim 3.

5. A compound or salt thereof according to claim 3 wherein:
Z₂ is trifluoromethyl; and
R₁ is as defined in claim 3.

6. A compound or salt thereof according to claim 4 wherein:
R₁ is phenyl and
Z₂ is trifluoromethyl.

7. A compound or salt thereof according to claim 1 wherein A=A₂ and R₃ is H and thus the compound has a structure represented by general formula III: 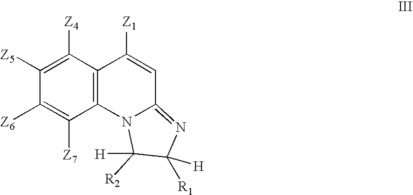
wherein R₁, R₂, Z., Z₄, Z₅, Z₆ and Z₇ are as defined in claim 1.

8. A compound or salt thereof according to claim 7 wherein Z₄, Z₇, and R₂ are all H and R₁ is selected from the group consisting of branched or unbranched alkyl, cycloalkyl, carboaryl and heteroaryl, each of which can optionally be substituted with one or more substituents independently selected from the group consisiting of halogen, cyano, nitro, acyl, oxycarbonyl, aminocarbonyl, alkylsulfonyl, arylsulfonyl, aminosulfonyl, hydroxy, acyloxy, alkoxy, aralkoxy, alkylthio, arylthio, aralkylthio, dialkylamino, cycloamino, carboxamido, sulfamido, trialkylsilyl, alkyl, aralkyl, perhaloalkyl, including, but not limited to, trifluoromethyl, alkenyl, alkynyl, carboaryl, heteroaryl, carbocyclyl, heterocyclyl; and
Z₁, Z₅, and Z₆ are independently selected from the group consisting of (i) unsubstitutable substituents: halogen, cyano, nitro, and (ii) substitutable substituents: hydrogen, acyl, oxycarbonyl, aminocarbonyl, alkylsulfonyl, arylsulfonyl, aminosulfonyl, carboxamido, sulfonamido, perhaloalkyl, including, but not limited to, trifluoromethyl, carboaryl, heteroaryl, carbocyclyl, heterocyclyl; wherein the substitutable substituents can optionally be substituted with one or more substituents independently selected from the group consisiting of halogen, cyano, nitro, acyl, oxycarbonyl, aminocarbonyl, alkylsulfonyl, arylsulfonyl, aminosulfonyl, hydroxy, acyloxy, alkoxy, aralkoxy, alkylthio, arylthio, aralkylthio, dialkylamino, cycloamino, carboxamido, sulfonamido, trialkylsilyl, alkyl, aralkyl, perhaloalkyl, including, but not limited to, trifluoromethyl, alkenyl, alkynyl, carboaryl, heteroaryl, carbocyclyl, heterocyclyl; and
wherein Z₅ and Z₆ can optionally form a carbocyclic, heterocyclic, aromatic or heteroaromatic cyclic structure containing 5 to 7 members wherein any member of the cyclic structure can optionally be substituted with one or more substituents independently selected from the group consisiting of halogen, cyano, nitro, acyl, oxycarbonyl, aminocarbonyl, alkylsulfonyl, arylsulfonyl, aminosulfonyl, hydroxy, acyloxy, alkoxy, aralkoxy, alkylthio, arylthio, aralkylthio, dialkylamino, cycloamino, carboxamido, sulfamido, trialkylsilyl, alkyl, aralkyl, perhaloalkyl, including, but not limited to, trifluoromethyl, alkenyl, alkynyl, carboaryl, heteroaryl, carbocyclyl, heterocyclyl.

9. A compound or salt thereof according to claim 8 wherein:
Z₁, Z₄, Z₆, Z₇, and R₂ are all H; and
R₁ and Z₅ are as defined in claim 8.

10. A compound or salt thereof according to claim 9 wherein:
R₁ is a phenyl group optionally substituted with one or more (up to 5) substituents independently selected from the group consisiting of halogen, cyano, nitro, acyl, alkoxycarbonyl, mono- and dialkylaminocarbonyl, alkylsulfonyl, arylsulfonyl, mono- and dialkylaminosulfonyl, hydroxy, acyloxy, alkoxy, aralkoxy, alkylthio, arylthio, aralkylthio, dialkylamino, cycloamino, carboxamido, sulfamido, trialkylsilyl, alkyl, aralkyl, trifluoromethyl, carboaryl, heteroaryl; and
Z₂ is as defined in claim 9.

11. A compound or salt thereof according to claim 9 wherein Z₂ is halogen and R₁ is as defined in claim 9

12. A compound or salt thereof according to claim 9 wherein:
R₁ is phenyl; and
Z₂ is chlorine.

13. A compound or salt thereof according to any of claims 1 through 12 inclusive wherein the compound is present in at least 90% enantiomeric excess of either the R or S configuration of said compound.

14. A compound or salt thereof according to claim 1 wherein A=A₂ and Z₁, Z₄, Z₅, Z₆, Z₇, R₂, and R₃ are all H, and the compound is present in at least 90% enantiomeric excess of either the R or S configuration of said compound and wherein the compound is selected from the group consisting of: 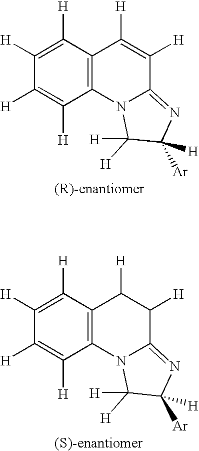
wherein Ar (representing R₁) is selected from the group consisting of Phenyl, 4-fluorophenyl, chlorophenyl, and 2-naphthyl.

15. A method for producing an enantiomeric excess of a chiral compound from a composition containing a racemic chiral substrate or further enhancing an enantiomeric excess of an already enantiomerically enriched chiral substrate or a meso-compound comprising the steps of:
contacting the chiral substrate with at least one DHIP or DHIQ compound or its salt or other derivative; under effective conditions;
reacting the compound or salt with the substrate at a catalytically effective temperature;
reacting the compound or salt with the substrate for a catalytically effective period of time; and
isolating the enantiomerically enriched or enantiopure compounds after the reaction involving the DHIP or DHIQ compound.

16. The method of claim 15 wherein the DHIP derivative is a compound of claim 1.

17. The method of claim 15 wherein the DHIP Derivative is a compound of claim 2.

18. The method of claim 15 wherein the DHIP derivative is a compound of claim 3.

19. The method of claim 15 wherein the DHIP derivative is a compound of claim 4.

20. The method of claim 15 wherein the DHIP derivative is a compound of claim 5.

21. The method of claim 15 wherein the DHIP derivative is a compound of claim 6.

22. The method of claim 15 wherein the DHIQ derivative is a compound of claim 7.

23. The method of claim 15 wherein the DHIQ derivative is a compound of claim 8.

24. The method of claim 15 wherein the DHIQ derivative is a compound of claim 9.

25. The method of claim 15 wherein the DHIQ derivative is a compound of claim 10.

26. The method of claim 15 wherein the DHIQ derivative is a compound of claim 11.

27. The method of claim 15 wherein the DHIQ derivative is a compound of claim 12.

28. The method of claim 15 wherein the chiral substrate is contacted with at least one compound or its salt of claim 14.

29. The method of claim 15 in which the chiral substrate has the formula: 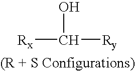
wherein Rₓ and R_{y} are different, and are independently varying carbon atom containing moieties and neither Rₓ nor R_{y} can be equal to H; and
wherein Rₓ and R_{y} both can be independently selected from the group consisting of substituted or unsubstituted aryl; substituted or unsubstituted heteroaryl; substituted or unsubstituted aralkyl; substituted or unsubstituted bi-, tri- or polycyclic aromatic system; substituted or unsubstituted, branched or unbranched, linear and cyclic forms of alkyl, alkenyl, and alkynyl.

30. The method of claim 15 in which the chiral substrate has the formula: 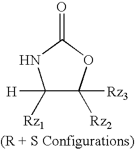
wherein R_{z1} cannot be H and R_{z2} and R_{z3} can be H and in addition R_{z1}, R_{z2} and R_{z3} are independently selected from the group consisting of substituted or unsubstituted carboaryl; substituted or unsubstituted heteroaryl; substituted or unsubstituted aralkyl; substituted or unsubstituted, branched or unbranched, linear and cyclic forms of alkyl, alkenyl, and alkynyl.
